(19) United States
(12) Patent Application Publication
(10) Pub. No.: US 2022/0203014 A1
(43) Pub. Date: Jun. 30, 2022

(54) WOUND THERAPY SYSTEMS

(71) Applicant: J&M Shuler Medical Inc., Athens, GA (US)

(72) Inventor: Michael Simms Shuler, Athens, GA (US)

(21) Appl. No.: 17/536,808

(22) Filed: Nov. 29, 2021

Related U.S. Application Data
(60) Provisional application No. 63/118,825, filed on Nov. 27, 2020.

(51) Int. Cl. A61M 1/00; A61F 13/00

(52) U.S. Cl. CPC A61M 1/90; A61F 13/00063; A61M 2205/3344; A61M 2202/0208; A61F 13/00068

(57) ABSTRACT

Systems, devices, and methods related to wound therapy are disclosed. Different aspects of wound care, including mechanical wound therapy, wound monitoring, irrigation, debridement, and delivery of therapies to the wound surface can be combined to improve effectiveness of treatment. The disclosed techniques can provide various type of clinical applications of wound therapies, including reverse pulse lavage, gas therapy, bacterial count measurements, pressure-based ulcer prevention, pain management, peritoneal dialysis, and controlled tissue in-growth, among others. In some instances, the systems described herein can be made portable and operable without the use of electricity, which provides potential to provide mechanical wound therapy in settings without access to extensive clinical facilities.

### CROSS-REFERENCE TO RELATED APPLICATION

This application claims the benefit of U.S. Provisional Patent Application No. 63/118,825, filed Nov. 27, 2020, which is incorporated herein by reference in their entirety.

### TECHNICAL FIELD

The present invention relates to patient wound care, and more specifically to systems and methods of wound treatment, delivery of medication, coverings and wound interface components.

### BACKGROUND

Mechanical wound therapy is a type of treatment used by physicians to promote the healing of acute or chronic wounds. For example, sealed wound dressings can be connected to a vacuum pump and placed onto an open wound for applying sub-atmospheric pressure to the wound. Such types of applications can be used to draw out fluid from the wound and increase blood flow to a wound area. One type of mechanical wound therapy is negative pressure wound therapy (NPWT), where negative pressure can also be used to pull medications, gas, fluids, biological tissues across the surface of the wound.

### SUMMARY

Various embodiments disclosed herein are drawn to wound therapy systems. The embodiments combine different aspects of wound care, including mechanical wound therapy, wound monitoring, irrigation, debridement, and delivery of therapies to the wound surface. The systems described herein can be applied to provide various type of clinical applications of wound therapies, including reverse pulse lavage, gas therapy, bacterial count measurements, pressure-based ulcer prevention, pain management, peritoneal dialysis, and controlled tissue in-growth, among others. In some instances, the systems described herein can be made portable and operable without the use of electricity, which provides potential to provide mechanical wound therapy in settings without access to extensive clinical facilities. For example, certain systems disclosed herein can be used in remote settings (e.g., battlefields or mass casualty settings) or developing countries without necessitating access to, for instance, wall vacuum, wall power source, or filtration devices. Through self-contained designs, these systems create the ability to provide mechanical wound therapy in various circumstances where effective wound treatment is often cumbersome and challenging.

In one general aspect, a mechanical wound therapy system includes a wound interface component configured to be positioned adjacent to a wound. The system also includes a vacuum source configured to generate a suction force that produces a negative pressure differential nearby the wound. An inflow component is fluidly coupled to the wound interface component and the vacuum source. A vacuum regulator device fluidly is coupled to the vacuum source. The suction force generated by the vacuum source is regulated and a set of parameters associated with the regulated suction force is monitored.

The system can include the one or more optional features. For example, in some implementations, the system includes a tensioning device configured to be placed adjacent to the wound.

In some implementations, the vacuum regulator device includes a microprocessor that regulates the suction force generated by the vacuum source and monitors the set of parameters associated with the regulated suction force. The vacuum regulator device also includes a communication module configured to transmit, for output, data representing the set of parameters monitored by the processor.

In some implementations, the communication module includes a near-field communication module. The near-field communication module is configured to establish a short-range connection with a computing device that is within a proximity to the apparatus, and transmit, over the short-range connection, the data representing the parameters to the computing device.

In some implementations, the communication module includes a Wi-Fi module.

In some implementations, the communication module or encrypts or otherwise secures the information being transmitted.

In some implementations, the Wi-Fi module is configured to connect to a local area network, and transmit, over the local area network, the data representing the parameters to a computing device connected to the local area network.

In some implementations, the Wi-Fi module is configured to connect to a wide area network, and transmit, over the wide area network, the data representing the parameters to a server that is remote from the apparatus.

In some implementations, regulation of the suction force applied by the vacuum source is programmable by a user.

In some implementations, the wound interface component, the vacuum source, and the vacuum regulator each comprise circuitry configured to be in data communication with a remote monitoring system.

In some implementations, the circuitry of each of the wound interface component, the vacuum source, and the vacuum regulator is configured to receive error data via a wireless signal to the remote monitoring system.

In some implementations, the wound interface component, the vacuum source, and the vacuum regulator each include at least one sensor.

In some the implementations, the system includes an exudate canister fluidly coupled between the wound interface component and the vacuum source. The exudate canister comprises circuitry configured to be in data communication with the remote monitoring system.

In some implementations, the system includes a remote monitoring system.

In some implementations, the vacuum source includes a portable vacuum.

In some implementations, the vacuum source includes a wall vacuum.

In another general aspect, a mechanical wound therapy system includes a dressing having a top layer and a bottom layer. The dressing is configured to be positioned adjacent to a wound, and the bottom layer is positioned to face the wound and includes a set of perforations. The system includes a vacuum source configured to generate a suction force that produces a negative pressure differential nearby the wound. A regulator device is fluidly coupled to the mechanical wound therapy system. The regulator device is configured to regulate the suction force generated by the vacuum source, and monitor a set of parameters associated with the regulated suction force.

The system can include the one or more optional features. For example, the vacuum regulator device includes a microprocessor that regulates the suction force generated by the vacuum source and monitors the set of parameters associated with the regulated suction force. The system also includes a communication module configured to transmit, for output, data representing the set of parameters monitored by the processor.

In some implementations, the communication module includes a near-field communication module. The near-field communication module is configured to establish a short-range connection with a computing device that is within a proximity to the apparatus, and transmit, over the short-range connection, the data representing the parameters to the computing device.

In some implementations, the communication module includes a Wi-Fi module.

In some implementations, the communication module or encrypts or otherwise secures the information being transmitted.

In some implementations, the Wi-Fi module is configured to connect to a local area network, and transmit, over the local area network, the data representing the parameters to a computing device connected to the local area network.

In some implementations, the Wi-Fi module is configured to connect to a wide area network, and transmit, over the wide area network, the data representing the parameters to a server that is remote from the apparatus.

In some implementations, regulation of the suction force applied by the vacuum source is programmable by a user.

In some implementations, the dressing, the vacuum source, and the vacuum regulator each include circuitry configured to be in data communication with a remote monitoring system.

In some implementations, the circuitry of each of the dressing, the vacuum source, and the vacuum regulator is configured to receive error data via a wireless signal to the remote monitoring system.

In some implementations, the dressing, the vacuum source, and the vacuum regulator each include at least one sensor.

In another general aspect, vacuum regulator apparatus for wound therapy includes an interface configured to be coupled to a vacuum source such that the vacuum applies a suction force to a wound when coupled to the interface. A processor is configured to regulate the suction force applied by the vacuum and monitor a set of parameters associated with the suction force applied by the vacuum. A communication module is configured to transmit, for output, data representing the set of parameters monitored by the processor.

In some implementations, the vacuum regulator is configured to be programmed by a user for regulation of the suction force applied by the vacuum source.

In some implementations, the set of parameters associated with the suction force applied by the vacuum source includes at least one user-specified parameter.

In some implementations, the device includes a rechargeable battery configured to power the processor and the communication module.

In some implementations, the communication module includes a near-field communication module. The near-field communication module is configured to establish a short-range connection with a computing device that is within a proximity to the apparatus, and transmit, over the short-range connection, the data representing the parameters to the computing device.

In some implementations, the communication module includes a Wi-Fi module.

In some implementations, the Wi-Fi module is configured to connect to a local area network, and transmit, over the local area network, the data representing the parameters to a computing device connected to the local area network.

In some implementations, the Wi-Fi module is configured to connect to a wide area network, and transmit, over the wide area network, the data representing the parameters to a server that is remote from the apparatus.

In some implementations, the communication module is configured to exchange bi-directional communications with one or more components of a negative pressure wound therapy (NPWT) system.

In some implementations, the one or more components include a wound interface component, an irrigation network, or an exudate cannister.

In some implementations, the device includes a storage device configured to store data representing the set of parameters.

In some implementations, the processor is configured to monitor device usage during a rental period for the vacuum regulator apparatus. The communication module is configured to transmit, for output to a billing system, data representing monitored usage of the vacuum regulator apparatus during the rental period.

In some implementations, the processor is configured to detect that the vacuum regulator apparatus has been turned on and being used for negative wound therapy. In response to detecting that the vacuum regulator apparatus has been turned on and being used for negative wound therapy, collect data indicating a patient identifier associated with the negative round therapy. In some implementations, the communication module is configured to transmit data representing the patient identifier for output to a billing system.

In some implementations, the device includes a microphone configured to collect utterances provided by a user. The processor is configured to process the utterances collected by the microphone to identify a voice query corresponding to the processed utterance, and generate an instruction to perform an operation based on the identified voice query.

In some implementations, the device includes a set of interface controls for adjusting settings for providing negative wound therapy to the wound.

In some implementations, the set of interface controls includes for providing negative wound therapy to the wound.

In another general aspect, a portable wound therapy system includes a reservoir module configured to collect and purify a fluid volume. A wound interface component coupled to the reservoir via a first tubing and configured to receive a portion of the fluid volume from the reservoir module, and provide the portion of the fluid volume to a wound. A pump module is coupled to the wound interface component via a second tubing and configured to generate a suction force that applies a negative pressure differential at the wound.

In some implementations, the reservoir module includes a collapsible vessel configured to collect rain or local water. A filter fluidly connected to the collapsible vessel and a purification component fluidly connected between the filter and the first tubing and configured to provide purified water to the wound interface component via the first tubing.

In some implementations, the filter includes a charcoal filter.

In some implementations, the filter includes a HEPA filter.

In some implementations, the purification component includes an ultraviolet light emitting diode configured to apply ultraviolet light to the rain or local water collected by the collapsible vessel.

In some implementations, the pump module includes a compressible collection canister coupled to the second tubing via a one-way valve, and a mechanical pump.

In some implementations, the pump module includes a rechargeable power source.

The some implementations, the compressible collection canister is coupled to a third tubing via a second one-way value. The third tubing is coupled to a collection bag.

In some implementations, the reservoir module includes a collapsible water collection cone.

In some implementations, the filter is configured to be positioned physically below the reservoir and the purification component is configured to be positioned physically below the filter such that fluid tends to flow from the reservoir through the filter and through the purification component under force of gravity.

In some implementations, the pump module includes a compressible enclosure. A mechanical spring positioned in the compressible enclosure and configured to bias the compressible enclosure to an expanded position. A first one-way valve is positioned on the enclosure and configured to allow flow from the wound interface component into the compressible enclosure. A second one-way valve is positioned on the compressible enclosure and configured to allow flow from inside of the compressible enclosure to a location exterior to the compressible enclosure.

In some implementations, the compressible enclosure is configured to expel fluid from the compressible enclosure when the compressible enclosure is manually compress and the compressible enclosure is configured to draw fluid into the compressible enclosure from the wound interface component when the mechanical spring expands the compressible enclosure.

In some implementations, the fluid volume includes a volume of irrigation fluid with a temperature below 15 C.

In another general aspect, a filtration apparatus for portable wound therapy includes a reservoir configured to collect a fluid volume. A filter fluidly is connected to the collapsible vessel. A purification component is fluidly connected to the filter and configured to purify a portion of the fluid volume. A tubing is configured to connect the purification component to a wound interface component.

In some implementations, the filter includes an activated carbon filter.

In some implementations, the purification component includes a deep ultraviolet light emitting diode configured to apply ultraviolet light to the portion of the fluid volume.

In some implementations, the reservoir includes a collapsible vessel configured to collect rain water.

In some implementations, the filter is configured to be positioned physically below the reservoir and the purification component is configured to be positioned physically below the filter such that fluid tends to flow from the reservoir through the filter and through the purification component under force of gravity.

In some implementations, the reservoir includes a collapsible water collection cone.

In another general aspect, a fluid purification apparatus for portable wound therapy includes a chamber configured to store a fluid volume. Tubing coupled to the chamber and is configured to control flow of a portion of the fluid volume from the chamber. A purification module is configured to purify the portion of the volume that flows from the chamber.

In some implementations, the chamber includes a single use or reusable injection intravenous bag.

In some implementations, the purification module includes an ultraviolet light emitting diode and a body.

In some implementations, the purification module positioned inside the injection intravenous bag.

In some implementations, the purification module is configured to be inserted into the injection intravenous bag such that the light emitting diode applies ultraviolet light to the portion of the fluid volume.

In another general aspect, a pump apparatus for wound therapy includes a body comprising a first plate and a second plate and defining a chamber. A first one-way valve couples the first plate to a first tubing. The first one-way valve is configured to permit flow in a first direction from the first tubing into the chamber in response to compression of the chamber. A second one-way valve couples the second plate to a second tubing, wherein the second one-way valve is configured to permit flow in a second direction from the camber into the second tubing in response to compression of the chamber.

In some implementations, the pump apparatus includes at least one spring inside the chamber and extending between the first plate and the second plate.

In some implementations, the negative pressure differential produced in the first tubing in response to compression of the chamber is within a range of approximately −25 mmHg to −200 mmHg.

In some implementations, the first tubing is configured to be coupled to a wound interface component placed on a wound. The second tubing is configured to be coupled to a waste chamber.

In some implementations, the pump apparatus includes an actuator configured to compress the chamber. A power source is configured to provide electricity to the actuator.

In some implementations, the power source includes a rechargeable battery.

In another general aspect, a pump for use with a wound therapy wound interface component. The pump includes a compressible enclosure, a mechanical spring positioned in the compressible enclosure and configured to bias the compressible enclosure to an expanded position, a first one-way valve positioned on the enclosure and configured to allow flow of fluid into the compressible enclosure, and a second one-way valve positioned on the compressible enclosure and configured to allow flow of fluid from inside of the compressible enclosure to a location exterior to the compressible enclosure.

In some implementations, the compressible enclosure is configured to expel fluid from the compressible enclosure when the compressible enclosure is manually compress and the compressible enclosure is configured to draw fluid into the compressible enclosure from a wound interface component when the mechanical spring expands the compressible enclosure.

In some implementations, the pump further includes a battery.

In some implementations, the pump further includes circuitry.

In some implementations, the pump further includes a battery, and circuitry configured to wirelessly communicate to a system other than the pump.

In some implementations, the pump further includes a motor assembly configured to compress the compressible enclosure.

In some implementations, the motor assembly is configured to compress the compressible enclosure according to an irrigation setting in which the compressible enclosure is repeatedly compressed with a time delay between compressions.

In some implementations, the time delay is five seconds.

In some implementations, the motor assembly is configured to compress the compressible enclosure according to a maintenance setting in which the compressible enclosure is compressed to a specified height in the expanded position.

In some implementations, the motor assembly includes a rod having a rail extending along a longitudinal axis of the compressible chamber, wherein a length of the rod corresponds to the height of the compressible chamber in the expanded position, a first compression plate coupled to one end of the rod, a second compression plate coupled to another end of the rod, a motor configured to move the first plate relative to the first plate along the rail, and one or more batteries configured to provider power to the motor.

In some implementations, the motor assembly is configured to positioned relative to the pump such that the first and second compression plates enclose a portion of the compressible chamber. The first compression plate includes a cutout for the first one-way valve and the second compression plate includes a cutout for the second one-way valve.

In some implementations, the motor assembly includes a first compression plate, a second compression plate, and an attachment module comprising a connector configured to be coupled to the first compression plate. One or more compression cords also each extend radially from the attachment module and terminate at a junction point on the second compression plate.

In some implementations, the motor assembly is configured to compress the compressible chamber by retracting the one or more compression cords into the attachment module such that respective lengths of the one or more compression cords from the attachment module to the junction point is shortened.

In some implementations, the motor assembly includes a manometer configured to measure suction force.

In some implementations, the manometer includes a manual manometer.

In some implementations, the manometer includes an automatic manometer.

In some implementations, the device includes a display component configured to present the suction force measured by the manometer.

In some implementations, the display component includes an analog pressure gauge.

In some implementations, the device further includes an alarm component configured to provide a wound care alarm based on the suction force measured by the manometer.

In another general aspect, a gas therapy system includes a gas tank containing a first gas, a wound interface component configured to be attached to a wound, a liquid reservoir containing a first liquid. The liquid reservoir is fluidly connected between the gas tank and the wound interface component such that the first gas can flow from the gas tank and through the first liquid to the wound interface component for treatment of the wound.

In some implementations, the first gas is nitrogen.

In some implementations, the first gas is chloride.

In some implementations, the first gas is oxygen.

In some implementations, the first gas is 100% oxygen.

In some implementations, the first liquid is saline.

In some implementations, the first liquid includes potable water.

In another general aspect, a wound therapy system includes a wound interface component, and a flowmeter fluidly connected to the wound interface component, wherein the flowmeter comprises a controller in data communication with a sensor. The controller is configured to output a first signal in response to the sensor sensing red blood cells.

In another general aspect, a wound therapy system includes a wound interface component, a collection system with a first zone containing first hydrophilic objects having a first size and a second zone containing second hydrophilic objects having a second size that is smaller than the first size. The system also includes an inlet port and an outlet port. The collection system is configured to be positioned between the wound interface component and a vacuum source with the inlet port fluidly connected to the wound interface component and the outlet port fluidly connected to the vacuum source such that fluid can flow through the inlet port, then through the first zone, then through the second zone, then through the outlet port under negative pressure being applied by the vacuum source at the outlet port.

In another general aspect, a wound therapy system includes a wound interface component, and a collection system with an inlet port and an outlet port. The system also includes a sponge having a plurality of sponge holes therethrough, wherein the sponge holes have a larger diameter near the inlet port than near the outlet port. The collection system is configured to be positioned between the wound interface component and a vacuum source with the inlet port fluidly connected to the wound interface component and the outlet port fluidly connected to the vacuum source such that fluid can flow through the inlet port, then through the first zone, then through the second zone, then through the outlet port under negative pressure being applied by the vacuum source at the outlet port.

In some implementations, a wound therapy system includes a wound interface component having a top and a bottom. The wound interface component is clear or sufficiently translucent between the top and the bottom. The system also includes a UV light source configured to be positioned above the top and shine UV light through the wound interface component to a wound positioned below the bottom.

In some implementations, a method of treating a closed wound. The method includes positioning a wound interface component on top of the closed wound, and flowing a gas from a gas supply source through the wound interface component to the closed wound and out of the wound interface component.

In some implementations, a wound interface component includes a top layer configured to substantially seal a wound, and a bottom layer having a silicone wound contact surface, wherein the silicone wound contact surface is roughened to encourage tissue ingrowth.

In another general aspect, a kit includes a wound interface component and a second wound interface component having a second top layer configured to substantially seal the wound and a second bottom layer having a second silicone or thermoplastic elastomer wound contact surface, wherein the second silicone or thermoplastic elastomer wound contact surface is smoother than the wound contact surface of the wound interface component to discourage tissue ingrowth.

In some implementations, a method includes first, applying a first wound interface component to a wound, wherein the first wound interface comprises a first silicone or thermoplastic elastomer wound contact surface that is roughened to encourage tissue ingrowth. Second, the method includes removing the first wound interface component from the wound after 1-3 days. Third, the method includes applying a second wound interface component to the wound, wherein the second wound interface component comprises a second silicone wound contact surface that is smooth to discourage tissue ingrowth. Fourth, the method includes removing the second wound interface component from the wound after more than 3 days.

In some implementations, a wound interface component includes a top layer configured to substantially seal a wound, a bottom layer having a wound contact surface, a first surface coating applied to the wound contact surface of the bottom layer of the wound interface component, and a second surface coating applied to the wound contact surface of the bottom layer of the wound interface component over the first surface coating.

In some implementations, a kit includes a first wound interface component including a first top layer configured to substantially seal a wound, a first bottom layer having a first wound contact surface, a first surface coating applied to the first wound contact surface of the first bottom layer of the first wound interface component. The second wound interface component includes a second top layer configured to substantially seal the wound and a second bottom layer having a second wound contact surface. A second surface coating is applied to the second wound contact surface of the second bottom layer of the second wound interface component, wherein the second surface coating is different than the first surface coating.

In another general aspect, a wound interface component includes a top layer configured to substantially seal a wound, a second layer positioned under the top layer, and a skin graft positioned under the second layer, wherein the skin graft is configured to release from the second layer and graft to the wound over time.

In another general aspect, a system includes a wound interface component, and a vacuum source fluidly connected to the wound interface component via tubing.

In another general aspect, a interface component includes a covering layer with a first side positioned away from a wound, a vacuum interface chamber defining an internal space in communication with a plurality of openings for distributing negative pressure from a vacuum source, where the vacuum interface chamber is positioned below the covering layer, and a porous dressing component positioned below the covering layer and being configured to cover the wound.

In another general aspect, a wound therapy system for use in treating a wound includes a wound interface component having a base layer having a wound contact surface. The system also includes a tensioner and an inflatable bladder. The inflatable bladder is positioned between the tensioner and the base layer.

In another general aspect, a separating system includes a body defining a chamber. The chamber includes a first separation partition with a first set of absorbent objects having a first size, and a second separation partition with a second set of absorbent objects having a second size. The first size is different from the second size, and the first separation partition and the second separation partition are fluidly connected to each other.

In another general aspect, a ultraviolet light sleeve includes a body defining a pouch, a refillable bag to be placed inside the pouch and configured to store a fluid volume, a light source configured to provide ultraviolet light to the fluid volume, and a battery configured to provide power to the light source.

In another general aspect, a mechanical wound therapy system is used for peritoneal dialysis. The system includes a wound interface component configured to be placed inside an abdominal cavity, an inflow tube fluidly connected to the wound interface component and configured to provide dialysis fluid into an area near the abdominal cavity, and an outflow tube fluidly connected to the wound interface component and configured to extract excess fluid from the area near the abdominal cavity.

In another general aspect, a mechanical wound therapy system is used for pain management. The system includes a wound interface component configured to substantially seal a wound and a first tube fluidly connected to the wound interface component and configured to provide local anesthesia to an area near the wound through the wound interface component. A second tube fluidly is connected to the wound interface component and configured to apply a suction force to the area near the wound.

In another general aspect, a barrier is used for pressure ulcer therapy. The barrier includes a base layer defining a plurality of perforations through the base layer, wherein the plurality of perforations are positioned, sized, and configured to allow flow, wherein the base layer define a top surface and a bottom surface. Top surface structures are positioned on the top surface of the base layer, wherein the top surface structures are positioned, sized, and configured to space porous foam material away from the perforations of the base layer when porous foam material is positioned on top of the barrier after the barrier is positioned in the wound. Air bladder structures are positioned on the top surface of the base layer, wherein the air bladder structures are configured to be inflated to provide cushioning along a surface of the wound.

In another general aspect, a mechanical wound therapy and therapeutic fluid delivery system includes a wound interface component configured to be positioned adjacent to a wound, a vacuum source configured to generate a suction force that produces a negative pressure differential nearby the wound, an inflow component fluidly coupled to the wound interface component and the vacuum source configured to allow control inflow of a therapeutic fluid, a regulator device fluidly coupled to the mechanical wound therapy and therapeutic fluid delivery system. The regulator device is configured to regulate the suction force generated by the vacuum source, regulate the rate and amount of therapeutics fluid inflowing through the inflow component, and monitor a set of parameters associated with regulation of the suction force and the rate and amount of therapeutic fluid inflowing through the inflow component.

Other features, aspects and potential advantages will be apparent from the accompanying description and figures.

### BRIEF DESCRIPTION OF THE DRAWINGS

FIG. 1 shows an example of an electronic vacuum regulator (EVR) system.

FIGS. 2A-2F show examples of a portable irrigation fluid collection and filtration system.

FIG. 3 shows an example of an irrigation platform.

FIGS. 4A-4D show an example of a non-electric pump for mechanical wound therapy system.

FIGS. 5A and 5B show examples powered pumps for mechanical wound therapy system.

FIG. 6 show an example of a gravity-independent mechanical wound therapy canister system.

FIGS. 7A-7E show an example of tensioning-bladder combination device.

FIGS. 8A and 8B show an example of a barrier device.

FIG. 9 shows an example of a suture wound interface component.

FIG. 10 shows an example of a portable mechanical wound therapy system.

### DETAILED DESCRIPTION

### A. Overview

The technology disclosed herein generally relates to systems, devices, and methods for wound therapy such as drug delivery and/or mechanical wound therapy (e.g., negative pressure wound therapy, or NPWT) as well as prevention of wounds and management of burns or other skin conditions. Systematic management and monitoring of traumatic or systemically ill patients or animals in a veterinary medicine setting can be performed. Such systems can be human controlled and/or autonomously controlled (e.g., using one or more computing devices) with pattern recognition and/or machine learning software to identify individualize practices for wound care. Autonomously controlled wound care systems can employ models trained using machine learning and artificial intelligence methods based on training data collected from previous patients or from the specific patient being treated. Mechanical wound therapy can be used to improve the management of open wounds from trauma or disease, and benefits from application of the features disclosed herein. From the time the wound is created it is beneficial if several interim activities occur prior to the final step in wound care, definitive soft tissue management. These interim activities often include irrigation and debridement, minimization of microbial load, delivery of therapeutics to the wound surface, monitoring of the wound and sequential approximation of the wound (closing of the wound).

Though wound care systems are often dependent on provider-directed wound care, a number of features discussed below can be used to improve wound care through development of robust integrated systems. By automating these interventions, a new mechanism of wound management has been described referred to as mechanical wound therapy. Various embodiments disclosed herein serve to improve patient care from the time an adequate irrigation and debridement of the wound is completed until the wound is ready for delayed primary closure, skin graft, or other means of definitive reconstruction.

This disclosure contemplates mechanical wound therapy systems, apparatuses, and techniques that integrate wound care elements to provide various unified approaches to wound care. As described herein, “traditional wound therapy” generally refers to a form of wound debridement and care using a procession of moist to wet dressing to cause non-selective debridement of necrotic tissue in a healing wound. Wound care has evolved from the rudimentary elements of “traditional wound therapy” to employ “active” wound care. Active wound care refers to the application of devices to a wound to actively change the environment in a positive manner that improves wound healing. One example of active wound care is negative pressure wound therapy (NPWT). NPWT can be an effective means of active wound care with certain limitations. The systems and techniques disclosed herein address such limitations by providing various means of active wound care that incorporates automatable elements of wound care into a single integrated system, which is referred throughout as “mechanical wound therapy” (MWT). These elements include NPWT, but also include and are no limited to irrigation, wound monitoring, therapeutic delivery, wound tensioning/approximation and autonomous systematic wound care. One of the numerous advantages of mechanical wound therapy is that the applicability or suitability of NPWT is sometimes limited to specific types of wound care, while mechanical wound therapy can be applied to broader categories of wound care.

In some implementations, a mechanical wound therapy system includes an integrated architecture that includes a regulated vacuum source and various modules (or components) of care. Such modules include a dressing/wound contact layer, an irrigation circuit, a tensioning device, among others. The modules can be systematically controlled and monitored to work in concert to provide automated wound care that is individualized to with respect to a particular patient and/or a particular wound type and chronicity. As discussed in detail below, the integrated architecture provides various advantages to wound care quality. As one example, the mechanical wound systems provide the ability to continuously process different types of wound care data that is measured by some or each of the modules. This processing techniques can be monitored in relation to patient outcomes to identify best practices in wound care. As another example, the mechanical wound care systems can use various automation techniques to recursively process data to evaluate measured data with minimal or no user input.

In various implementations, the mechanical wound systems are configured as connected platforms that advantageously use interconnectivity between modules to automate several aspects of clinical decision-making. For example, sampling of the wound surface can indicate the need for additional treatments such as antibiotics or growth factors or other medications. The timing of the wound (acute, sub-acute or chronic) as well as even the number of days after wound creation can be used to guide management. Monitoring of tension on the wound tensioner can be monitored to increase or decrease tension on the skin edges. Tension and wound separation can also be used to determine the duration and volume of the unidirectional bladder/tensioner. Wound and blood metabolites can be used to induce managements such as anti-inflammatories, stem cells, hyperbaric/concentrated oxygen, growth factors, pain managements as well as other interventions.

Additionally, systematic monitoring and interventions can be incorporated into the system and be automated to provide tailored autonomous therapy. Temperature, arterial pressures, growth factor monitoring through blood access through and IV or arterial line can be incorporated. Systemic medications can be administered and controlled via the IV or arterial line in order to promote both wound healing and systemic whole patient health. Near infrared Spectroscopy (NIRS) and other noninvasive physio-monitors can be used to monitor local or systemic perfusion at the wound area or other remote areas. Respiratory function can be monitored through a pulse-ox, NIRS, arterial lines, blood carbon dioxide levels or other means. These signs can be used to monitor levels of consciousness, signs of sepsis as well as concerns for over medication in areas such as depressants like narcotics. Interventions such as antidotes can be automatically scheduled such as naloxone for narcotic overdose.

Blood pressure and organ perfusion can be monitored via pH monitoring, NIRS, arterial lines or other monitored in order to regulate systemic interventions such as intravenous fluids, insulin, glucose, pressors, anti-inflammatories or other modalities.

Patient active feedback can be incorporated to guide management such as pain scale input from the patient can guide modalities such as wound tensioning, or local anesthetics or systemic analgesia such as with patient-controlled analgesic (PCA). An algorithm can be designed to incorporate patient feedback on other factors such as temperature, leaks in a seal, increasing/decreasing swelling, signs of sepsis, decreased respiratory rate/effort.

Additionally, the mechanical wound systems can be configured to use various types of recognition techniques to identify patterns relating to wound care. As one example, the mechanical wound systems can use machine learning models to classify wound care data based on statistical information or knowledge gained from patterns and their representation in training data sets. The mechanical wound systems can be configured to employ different types of machine learning models, such as statistical techniques, structural techniques, template matching, neural networks, fuzzy models, hybrid models, among others. For example, wound chronicity (days from injury), mechanism of injury (sharp, blast, burn, pressure injury) can be incorporated to set expected standard wound characteristics over a period of time. Wound bacterial colonization and speciation can be monitored as well as metabolites for certain bacteria can be sampled such as used in urinalysis (luekoesterase and nitrites or other factors). Ultraviolent (UV) light could be used through the translucent patient contact layer or be incorporated into the layer in order to provide wound cleansing without the use of antimicrobials. Additionally, chemical antimicrobials such as soaps, disinfectants, alcohol, hydrogen peroxide, betadine can be administrated and flushed afterwards in order to prevent extended exposure. The frequency and duration can be controlled by the system. Wound sealants can be applied to the surface of the wound through the dressing/wound contact layer.

In some other implementations, the mechanical wound systems can be configured to use various types of artificial intelligence to improve wound care. Using the integrated architectures disclosed herein, the system can process data measured and/or collected by individual modules to, for example, identify rashes and lesions, measure and analyze wounds, provide colorimetric testing of wound images, or classify the severity of wounds. For example, NIRS can be used to define perfusion. pH monitoring can be used fr perfusion or bacterial infection. Ultrasound can be incorporated to monitor perfusion, depth of granulation tissue, abscess formation or even for inducing healing in the manner such as bone stimulators using ultrasound or electromagnetic stimulation. Modalities such as electromagnetic fields or ultrasound can be used to stimulate bone healing in associated bony injuries that are common with traumatic wounds. These modalities can be separate devices under the control of the mechanical wound therapy device, or they can be integrated modules, either unique or incorporated into advanced versions of the wound dressing. The systematic autonomous control feature of mechanical wound therapy can be used to control multiple modules of the system and receive inputs, provide outputs to accessory devices not related to the mechanical wound therapy device, but thereby placed under the control of the MWT device. In this fashion, the MWT device can serve as a control unit for both the intrinsic modules and extrinsic accessories. The initiation and scheduling of the therapies can be managed through the artificial intelligence (AI) system that would allow for specific fracture type, location and fixation management used. These inputs can be programable and used to tailor management.

Control of the system can be managed at the bedside as well as remote. The system can be connected to the electronic medical record to automatically record the data obtained as well as the interventions provided via a time stamp. The algorithm decision justification can be defined in the medical record. Response to therapy can de documented and reviewed as well as learned by the system. Remote access can be utilized either by others in a medical center as well as clinicians across the country or world. Bar codes or radiofrequency identification (RFID) can be utilized to easily record manual interventions provided by nurses or other health care providers. The system can be managed by computers, smart devices or other control systems such as voice control or other modalities.

The system will be interactive both to external sources such as an electronic medical record or outside medical providers as well as internal communication and feedback. Internal communication would be set up between all the modalities. These modalities would be items such as the wound or patient contact layer, the tensioner, the unidirectional bladder, the arterial lines, pulse ox, NIRS sensors, pH monitor, thermometer, metabolite sensors, ultrasound, electromagnetic fields or other input monitors. These components can be powered by batteries or wired power sources. Body heat, solar power and body motion can be used to power modalities. System r component initiation to preserve power or battery life can be started by manual switches, peeling off a backer or body heat or electrical signals associated with normal physiologic signals, RFID signaling or other means. Batteries can be rechargeable or disposable and can be solar charged.

Communication can be via wired communication or wireless, Bluetooth or other novel communication modalities that can be under secure or encrypted to protect personal data. Feedback between system components will be utilized to drive algorithms and learning based on expected criteria. For example, if leukoesterase or nitrites are sampled on the wound surface, local wound antibiotics or irrigation can be administered. If continual bacterial evidence is detected, additional interventions such as systemic/IV antibiotics can be administered or recommended to clinicians or even dressing removal and formal irrigation and debridement can be recommended for ideal management. Additionally, based on metabolites or specific factors bacterial identification and even specificities can be determined in order to recommend ideal antibiotic use.

Laboratory findings such as nutritional factors to include but not limited to serum proteins (transferrin/albumin/prealbumin/retinol-binding protein or others) and other indicators can be used in order to guide nutritional needs and recommendations for dietary planning in order to promote global healing.

Unique identifiers can be both fashioned at time of manufacturing as well as programable identifies can be programed in order to monitor patients in a setting where multiple patients are being treated in the same facility. Identifiers can be used for different parts of the body as well as different patients. Each component in the system can be created with identifiers for the type of component as well as programable locations and patient identifiers. These mechanisms allow for a creation of a hospital wide system that allows for the management of multiple patients on a medical center or even nationwide system. These systems allow for remote monitoring of multiple patients for improved outcomes as well as for billing and reimbursement systems. Medical compliance and actual treatment compliance can be ensured. Improved research and therapy guidelines can be better created based on improved data collection. This system would allow for better assessment of actual interventions and responses/outcomes to these interventions. Objective and subjective data can be included such as patient assessment data and outcomes.

### B. Electronic Vacuum Regulator (EVR)

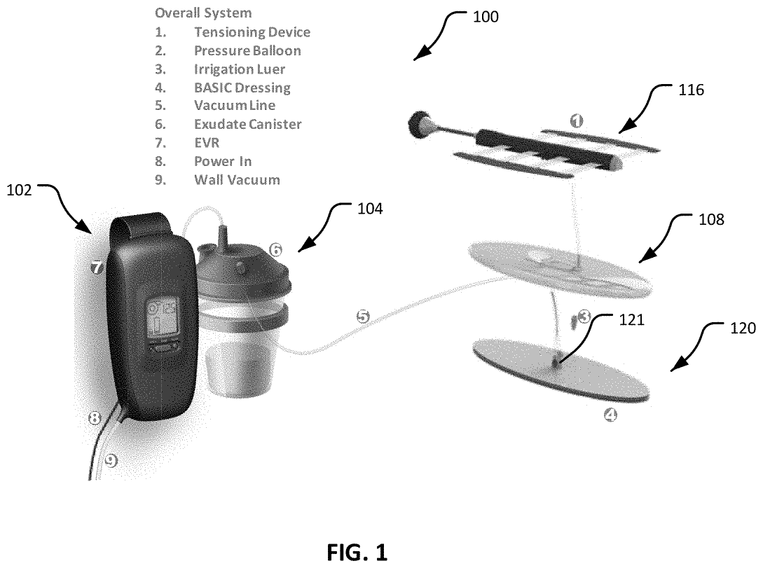

FIG. 1 shows an example of an electronic vacuum regulator (EVR) system 100 including an EVR 102, exudate canister 104, unidirectional bladder 108, tensioner 116, and unified wound interface component 120. The EVR 102 can reversibly affixable (e.g., locked) to one or more vacuum components. The EVR 102 includes two power sources. The first power source is a battery power source, such as primary cell battery (e.g., non-rechargeable), a secondary cell battery source (e.g., rechargeable), or a combination thereof. The second power source is a wired electrical connection (e.g., an electrical cord) suitable to receive electrical power from a static power source (e.g., a wall outlet) or a larger vacuum source to which it can connect.

The EVR 102 can include an integral or a separate vacuum unit (not shown in FIG. 1) configured to draw power from the first or second battery source. The vacuum unit allows the EVR 102 to operate in a portable state, e.g., not electrically connected to a wall outlet or to a primary vacuum source, for limited periods of time. The EVR 102 functions in combination with wound interface component 120, which can include a sealing layer, (e.g., hydrocolloid or other adhesive) described further herein. Such sealing layers reduce the vacuum pressure rate of decay within the wound interface component over time, for example, during periods in which vacuum (e.g., vacuum pressure) is not actively applied. In such examples, pump power used to maintain a threshold vacuum pressure within a therapeutic range is reduced, allowing for additional pumps to serve an intermediate (e.g., bridge) role for uses in portable applications. For example, a partially bed-bound hospital patient traveling to/from the bathroom.

The EVR 102 further includes a wireless communications array (e.g., Wi-Fi, Bluetooth®, cellular) for communicating with capable devices over a local or distributed network (e.g., local network, wide area network, cellular network, or internet). The EVR 102 includes communications protocols for wireless communication of information (e.g., data) obtained by one or more sensors of the EVR 102 measuring specific parameters. These parameters can include, but are not limited to, vacuum strength, air flow, fluid flow, or fluid volume. Additionally, information notifications can be transmitted by the EVR 102 communications array such as parameter values, parameter threshold alarms, or fault alarms. For example, alarm notifications such as unexpected increases in fluid flow rate or volume, as seen in patient bleed outs, are monitored and communicate alarm notifications to remote stations, such as nursing stations or distributed monitoring locations. Bi-directional communication can occur between the EVR and the different components of the wound care system. Commands from the EVR can activate or release the tensioner. Communication between the EVR and the unidirectional bladder can result in the bladder inflating, deflating or changing the sequence or speed of inflation and deflation. The EVR can communicate medication release timing, duration or rate based on feedback from the components or from external controls.

The communications array includes components capable of bi-directional communication of data and/or command structures. For example, to receive remote commands from networked devices, notification communication between local terminals (e.g., patient room to nurse station), or over wide area networks (e.g., a distributed data server, a centralized server group). In some embodiments, remote users may view data, change settings, view wound information, review treatment parameters, or monitor alarm notifications. Remote users can provide commands to the system to cease or initiate or continue treatments or modalities such as tensioning the wound, increasing bladder pressure or sequencing among other things.

The communications array can additionally transmit identification information (e.g., patient or consumable identification information) to customer service or billing centers for real-time assessment of function (to support trouble-shooting) and use (to support billing). A bar code or radiofrequency identification system can be incorporated to read or scan treatment modalities. The modalities can be scanned and initiated through the system. The modality application can be time stamped and entered into the medical record for treatment monitoring and confirmation that the modality was accurately administered in an accurate time frame. This notification can be disseminated across the entire communication system including remote users/providers.

The EVR can have a tracking mechanism using GPS or other location identification systems. This system can allow for location identification both on a map as well as within a building or system such as a hospital or business campus. It can provide altitude information in order to determine location based on which floor in a building a unit is located. A signaling beacon or identification chirp can be incorporated to identify the location within a room. A back up battery can be incorporated solely for this purpose in order to signal location even with a dead main battery. Signals can be sent to a specific central location monitoring system maintained by the manufacturer which can assist is determining the last recorded location of the device prior to battery depletion. Once activated, a new location can be determined similar to cell phones. This location monitoring can be centrally at the manufacturing site or established through an app or computer program that allows providers to monitor the location of its multiple units. As batteries start to run low, alarms can be signaled to locate and recharge the units. Monitoring for owners or distributors can be established for provider owned units similar to find my phone apps.

The EVR 102 includes one or more reversible linkages for temporary attachment to a second or more supplementary vacuum pump capable of delivering therapeutic vacuum pressure within a range of 0 to −250 mmHg. The supplementary vacuum pump is self-contained, powered vacuum-producing unit. In some embodiments, the supplementary vacuum pump is drop-resistant to prevent damage to the supplementary vacuum pump during transport and use with the EVR 102.

Optionally, the EVR 102 provides control features for the supplementary vacuum pump, such as power commands, function commands, through the EVR 102 digital display. In such optional embodiments, the EVR 102 provides control features for the supplementary vacuum pump and is maintained between the patient and supplementary vacuum pump therefore less biological material will enter into the supplementary vacuum pump. As such, one supplementary vacuum pump can provide concurrent or sequential vacuum pressure to one or more EVR 102. For example, a ward or care setting could include a limited number of portable secondary vacuum pumps, for use by multiple EVRs 102 during periods in which a patient requests prolonged mobility. The remainder of the time, the EVR 102 maintains vacuum pressure on the patient wound interface component internal or external power and suction (e.g., vacuum pressure).

Both the EVR and the supplemental (larger unit) can have the capacity to either reverse the direction or suction in order to provide bursts or sustained positive pressure. This positive pressure can also be created with a separate pump. Positive pressure can be used in the reverse pulse lavage system in order to accelerate/accentuate flow changes to promote tissue cleaning and foreign body and dead tissue removal.

In some additional embodiments, the supplementary vacuum pump includes some internal control and data recording features, as well as bi-directional communication capabilities to communicate with the connected EVR 102. The EVR 102 and supplementary vacuum pump can optionally include a GNSS (Global Navigation Satellite System) sensor (e.g., GPS) for GNSS geolocation tracking capabilities. The data recording features include recording of EVR 102 status notifications including but not limited to battery life, attached canister pressure or content levels, seal information, leak information, or fluid flow data. Recorded data can be stored in memory components within the EVR 102 or remotely in a distributed computing environment (e.g., a cloud server).

In some embodiments, the EVR 102 and supplementary vacuum pump include specific identification numbers in order to allow tracking and memory of therapeutic activities for specific patients. There can be a permanent identification number, such as a serial code, and/or modifiable code that can be input or created by the clinician or staff or patient. Additionally, each modality, such as a wound interface component, tensioner, bladder or other components, can have both a permanent and modifiable identification number or name. Similarly, any therapeutic placed in the system can have a serial number in order to monitor for effectiveness, adverse events as well as for billing and documentation. The EVR 102 includes a scanning device (e.g., laser scanner, optical scanner) to read and/or record data via computer readable codes, such as barcodes. The computer readable codes can encode data such as medical record data, patient identification data, material identification data, or medical component identification data. Additionally, these data can be added to patient medical record monitoring in order to allow monitoring to be included in the medical record such as telemetry.

The EVR 102 can include line fittings for temporary connection to an external vacuum source. Examples of external vacuum sources include fixed vacuums at static locations providing constant vacuum pressures, e.g., in-wall or building vacuum lines, or portable vacuums such as a supplementary vacuum pump. Line fittings are manufactured to provide custom configurations for proprietary commercial and patient safety reasons. In most in-patient settings as well as operating rooms, in-wall vacuum pressure (e.g., wall suction) is present providing an available, unregulated vacuum pressure source. The EVR 102 line fittings function to plug into available wall suction. The EVR 102 line fittings can optionally be attached to a suction splitter, e.g., a device in which one EVR 102 provides vacuum pressure control to more than one suction circuit. As an example, a suction splitter connected to an EVR 102 regulates the vacuum pressure transmitted to more than one wound interface component. The wall suction or other vacuum source adapter can be removed or switched out in order to accommodate different locations and adapter requirements.

The EVR 102 includes pressure monitoring and regulation functions to monitor and regulate external vacuum sources. A pressure regulator functions to limit the magnitude of vacuum pressure allowed to maintain a specific therapeutic set point or range. An exemplary unregulated wall source will maintain a vacuum pressure between 250 mmHg and 500 mmHg. Functional applications for clinical use function between 0 mmHg and 250 mmHg and therefore the EVR 102 regulates vacuum pressure from unregulated wall sources to within the range of clinical use function, e.g., for a wound interface component 120 or a suction tube.

The EVR 102 can connect in series with unregulated vacuum sources, examples including a wall vacuum source in a hospital room, a portable powered vacuum source, or a manual or spring-action pump. The EVR 102 connects to unregulated vacuum sources through a functional appendage, such as tubing for suction, a wound interface component 120 for mechanical wound therapy, or other treatment component, such as a dressing. The EVR 102 operates as a control unit for the therapeutic delivery of vacuum pressure to a wound interface component 120 sealed over a patient wound. The EVR 102 operates to regulate unregulated vacuum sources, for example, the vacuum pressure magnitude of wall suction present in hospital settings.

The EVR 102 can include a display (e.g., a screen such as an LED screen) which functions as an interface for the user and EVR 102 functionality to display textual, numerical, or pictographic information to a user. The display can display information in any language stored on memory. The display can be a passive display with no user interaction capacity or an active display which the user can input information into directly (e.g., a touch screen). Displayed information can include more than one category of alarm notifications including a failure mode, or failure warning in textual information or numerical (e.g., a code, or number representation) for referencing in a user manual or reference sheet.

In some embodiments, the user inputs information into the display. Via this interface, the user inputs function parameters, or control structures program specific functions. Additionally pictures can be used to describe the alerts or failures in order to communicate the alert for persons of different languages or education status.

The EVR 102 communication array transmits information to remote computing devices (e.g., remote monitoring), for example, transmitting error code data for trouble shooting. In some embodiments, the EVR 102 includes components for temporary connection of portable memory (e.g., a memory card, a USB drive, an external hard drive) for copying data stored on the local memory to the portable memory. In some embodiments, information stored on the EVR 102 memory or hard drive is cryptographically encrypted. The encryption can comply with a national standard, e.g., HIPPA compliance.

In some implementations, the EVR can be voice-controllable and thereby be configured to process voice input in addition to (or alterative to) manual input. Voice control, in such implementations, can occur similar to voice control of smartphones, e.g., by processing voice queries received from providers or patients. Data or alarms of system feedback can be communicated to providers or patients as well in a bi-directional fashion. Such communications can be received or input from remote settings. Different languages can be activated based on desires both orally or written on the LED screens. Pictures or logos can be used to communicate to people unable to read.

The EVR 102 connections can include magnets to facilitate correct placement and positive alignment of attached components. In some embodiments, the connections can be manufactured in the form of geometric shapes to prevent components from connection at incorrect locations. In some embodiments, a passcode or login information can be used to lock (e.g., disable) or unlock (e.g., enable) the EVR 102. The connections can include communication components capable of transmitting information from the wound interface component 120, such as measured pressure, oxygenation, pH, ion levels or other blood chemistries, photo detectors, or antibody probes

The wound interface component 120 includes in memory threshold condition values to trigger the wound interface component 120 to perform preset therapeutics corresponding with threshold condition values. For example, unexpected increased fluid flow triggers coagulant release to the wound surface to potentially clot unexpected bleeding. The wound interface component 120 delivers medications such as thrombin or factor VII to the wound via a positive pressure delivery system. Additionally, if the tensioner 116 is in place, the wound interface component 120 can automatically, via direct or remote control, direct the tensioner 116 to provide compression over the wound thereby controlling bleeding or hemorrhage. In embodiments in which the unidirectional bladder is placed separately or as part of the tensioner 116 system, the bladder inflates to provide additional pressure on the bleeding wound.

Medication ampules can be designed to apply metered doses of medication over specific and preset intervals similar to a PCA. These ampules or syringes can be inserted into the EVR and specific regimens can be initiated based on provider or patient desires as well as preset parameters. These medications can be anesthetic, antibiotic, anti-inflammatory or other medications. The medication can be fluid, gas, powder, among others.

In some instances, medication or irrigation is colored to confirm complete wound coverage. The IV bags with irrigation can have a dissolvable dye in the liquid. This dye would be non-permanent so it would not create discoloration in the healed wound or skin. But it would allow for confirmation that the wound was completely irrigated.

The unified construction of the wound interface component 120 and sealing layer are composed of substantially transparent materials thereby allowing light emitted from bound probes at the wound surface to be detected by external wound interface component components. The wound interface component 120 includes photo-sensing devices to measure emitted light and algorithms to quantify detected information such as bacterial bioburden.

These modalities and treatments could follow a predetermined schedule or in reaction to a detected event, e.g., a high risk event such as sudden uncontrolled bleeding such as an acute vascular bleed beneath a NWPT wound interface component 120. The EVR 102 controls and integrates component response. The EVR 102 includes algorithms and/or other control structures to coordinate component responses and such responses are recorded. Alternatively, each component can have respective processors responding to information independent of the EVR 102.

The radial irrigation tubing can have a constriction centrally that offers some slight resistance. By putting a resistor centrally, this would ensure flow occurs in all directions even if the wound interface component is cut asymmetrically. Uneven flow may occur if one side is cut closer to the central suction chamber. With central constrictions, the resistance will be centrally. That resistance will resist flow more than the length of the tubing past the constriction. Therefore, uneven tubing lengths will not result in uneven flow.

Wound interface component 120 flow meters measure flow velocity and total fluid amount that has flowed through the wound interface component 120. Canisters 104 include mechanisms for measuring flow (e.g., float bobs that rise as fluid comes into the canister 104 and the rate at which this occurs can determine velocity). The EVR 102 can be programmed to include flow rate alarms, e.g., flow rates or total volumes exceeding a programmed threshold value. The EVR 102 receives the alarm status from the wound interface component 120 and records the occurrence of an alarm-triggering event. These algorithms will come preset, but can be customized through the touch screen on the EVR 102. Algorithms can be added to account for high flows during periods of wound irrigation. The EVR 102 can include a pause button (or irrigation button including anticipated volume of irrigation value) for temporarily ceasing the alarm status or response.

VAC assisted exsanguination is a known risk of NPWT wound interface components 120 with real-time flow meters. Flow meters can connect to any connection point in the vacuum circuit between the EVR 102 and the wound interface component 120. In some embodiments, connected flow meters measure absolute volume of flow and liquid content. As one example, the amount of hemoglobin present in effluent. As a second example, spectroscopy probes measure the specific chromophore amount in a fluid or tissue. The EVR 102 measures exudate composition to detect high flow through the system related to irrigation, for instance, the presence of concentration of hemoglobin described above (e.g. sudden drop in concentration of Hgb).

If increased flow rate and specific liquid characteristic are detected, the EVR 102 can cause components to perform corresponding functions automatically (e.g., without human interaction). For example, a spectroscopy sensor connected to the wound interface component 120 can detect the presence or concentration of red blood cells in exudate (e.g., fluid being evacuated from the wound). Detection of a red blood cell concentration value above a threshold in the exudate fluid being evacuated from the wound can be measured and recorded to prevent VAC assisted exsanguination as well as to monitor total volume input and output from the wound or wound interface component 120). Conditions such as cessation of suction trigger the EVR 102 to respond automatically. Additionally, therapeutics, such as a coagulation substrate, in self-contained vessels can be connected either to the wound interface component 120 or to the EVR 102 thereby enabling automatic delivery to the wound surface if a bleeding event is detected independent of human intervention.

Alternatively, if a change in exudate pH is detected or other indications of the development of an infection, the system 100 can be preset to deliver a preset amount of irrigation that can be premixed with antibiotics or other means.

In some instances, wound interface component 120 has a unified construction with a sealing layer that functions as a dressing for sealing a wound. The wound interface component, in such instances, is composed of substantially transparent materials that allow light emitted from bound probes at a wound surface to be detected. Additionally, the wound interface component 120, in such instances, does not include a dedicated irrigation circuit since delivery of fluid to the wound site is not required in these circumstances. The wound interface component 120 that functions as a dressing can be combined with one or more features of other embodiments described throughout this specification. For example, a wound therapy system can include a dressing with a top layer and a bottom layer. The dressing is configured to be positioned adjacent to a wound and the bottom layer is positioned to face the wound and includes a set of perforations. The system can include a vacuum source configured to generate a suction force that produces a negative pressure differential nearby the wound. The system also includes a regulator device fluidly coupled to the mechanical wound therapy system. The regulator device can be configured to regulate the suction force generated by the vacuum source, and monitor a set of parameters associated with the regulated suction force.

The EVR 102 is separate from attached vacuum sources enabling a logistical flexibility as EVR 102 can be stored in a Pyxis™ or other hospital inventory center. This small unit can be removed from static (e.g. counter/storage space) or automated (e.g. from the Pyxis™) and that event can trigger the start of a billed use (e.g., a rental charge) to a specific patient e. The EVR 102 can then be attached to a wall suction source for bed-bound or predominantly bed-bound patients or to a portable vacuum source for mobile patients or patients during periods of mobility. Due to this design flexibility, fewer EVRs 102 are needed to be stored at a location (e.g., hospital ward) than alternative, more expensive, large vacuum pumps in which the control unit and vacuum pump are fully integrated. This would lead to increased efficiencies in storage and billing.

This system 100 also records irrigation periods for billing purposes (e.g. to provide a record of the event for audits, or the ability to record irrigation events and details) and to monitor compliance of the patients, providers, or ancillary care person in accordance with prescribed rate and volumes of wound irrigation.

Additionally, the EVR 102 includes an internal timer and data storage device (e.g. SSD) such that the operation of any or all of included functions is automatically tracked with a corresponding record of timed use for accurate and documented billing (e.g., the Pyxis™ record). Alternatively, the EVR 102 communication capabilities updates a remote control station during real-time use of the device. The data storage device can be of different magnitudes and based on the size of the data storage device re-looping protocols can be implemented that overwrite stored data at a programmed time interval, or after a certain quantity of data has been collected.

In some implementations, the EVR is configured as a metered drug delivery system that allows for sustained delivery of medication similar to an implantable pain pump that delivers lidocaine to surgical sites. For example, the EVR can be similar to a patient-controlled analgesia (PCA) device in which therapy is delivered based on input received on provider or patient controls. This can be performed remotely as well. Metered and specific doses of analgesic, antibiotics, or other therapeutics can be delivered on demand. Regulatory parameters can be pre-established and even modified based on wound healing. The therapeutic agent can be fluid, gas, biologics or other means. Wound moisture can be monitored as well to prevent drying out of the wound as well as maceration of the wound to create an ideal environment.

The EVR can incorporate a catheter or IV system that allows for local or systemic delivery of medications or monitoring of local or system environments. This catheter(s) could be place in the soft tissue or the vasculature such as an IV or arterial line. Blood pressure, pulse or other vital signs can be monitored and recorded as well as hematological elements such as inflammatory marker or growth factors or other items.

The EVR based on programable algorithms could assist in directing care. If the bacterial load is detected to be increasing. Audible or written recommendations for irrigation or antibiotic use can be suggested to the provider or patient. Sensors such as Near Infrared Spectroscopy (NIRS) can be incorporated into the wound contact interface or the tensioner to monitor blood floor and well to insure ischemia does not occur under the tensioner. Additionally, UV light can be used to purify the irrigation fluids or even the wound surface in order to combat infections within the wound. These modalities can be monitored and activated based on wound conditions and system feedback or protocol.

The EVR can have a gas concentration & purification system that is able to create purified oxygen or other gases from the atmosphere. Additionally, chemical cartridges can be inserted into the EVR or the larger EVR housing that would allow the conversion of atmosphere air into desired gases/gas combination for use in the system.

The tensioner can also be used to offload the tension on the wound surface or in the case of a wound that is closed but is tight due to swelling or loss of skin. Suture or staples or other means are used to pull the skin edges together. Typically, in the traumatic setting the skin edges are damages and are prone to ischemia due to the over pull of the suture on the skin edges at the wound which can cause wound breakdown or dehiscence. If the tensioner is placed away from the skin edges or suture line and tension is pulled towards the center of the wound, the tension at the suture line can be reduced. The ribbons on the tensioner will be able to extend several inches. In this setting, the ribbons can be attached to the skin and anchored a safe distance from the wound and tension applied towards the wound. This application would reduce tension and ischemia at the wound by applying force a safe distance from the actual traumatized tissue that is trying to heal.

The EVR can also control a tourniquet that can be used in a trauma setting. Tourniquets are used in the setting of uncontrolled bleeding, to prevent blood loss. However there is a limit to the time a tourniquet can be used (2 hours) before permanent damage such as reperfusion injuries (compartment syndrome) or permanent ischemia/necrosis can occur. Automated tourniquet can allow for perfusion to be restored for limited time frames to extend tourniquet use. Tourniquet release with direct pressure on the wound during reperfusion can occur.

### C. Portable Filtration/Purification System

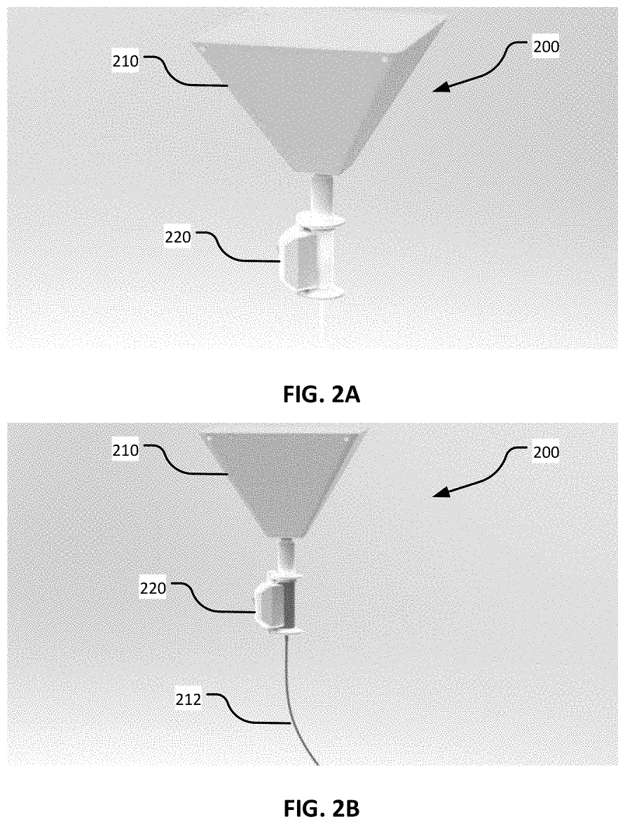

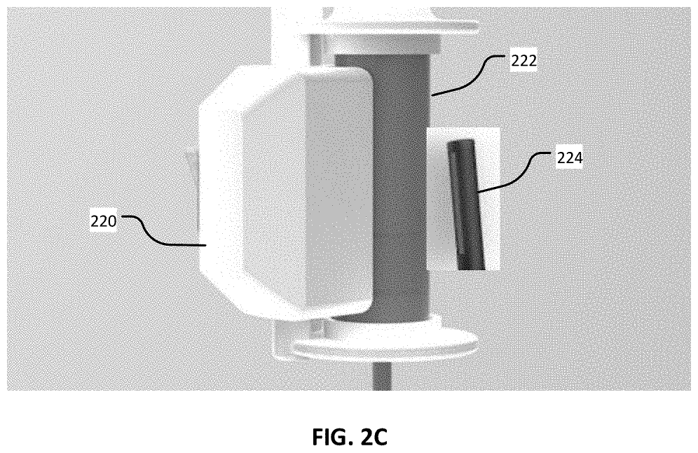

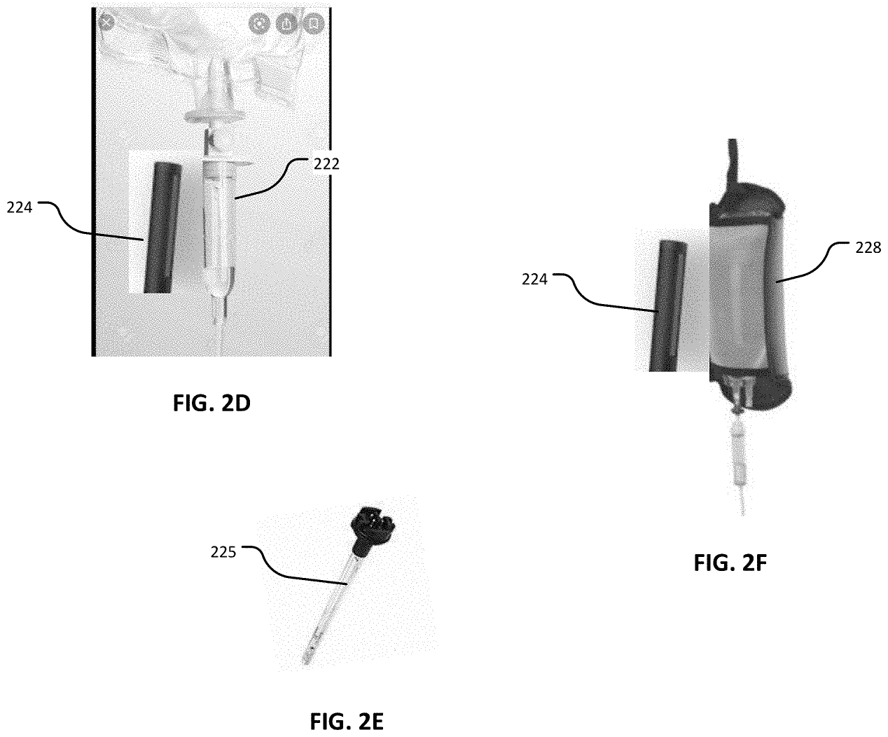

The system 100 can include a portable irrigation fluid collection and filtration system depicted in FIGS. 2A-2C. As shown in FIG. 2A, a system 200 includes a fluid collection device 210 and a filtration device 220. The fluid collection device 210 is composed of a flexible material (e.g., plastic, polymer, fabric) that allows the system to collapse. The fluid collection device 210 functions to collect and direct fluids into the filtration device 220. The system 200 can optionally include rigid poles for independent use, or can be hung from nearby standing structure such as trees.

The collection device 210 funnels fluid towards the filtration device 220 attached, reversibly or permanently, at the apex of the collection device 210 funnel. Fluid is directed from the collection device 210 towards the filtration device 220. Referring to FIG. 2B, the filtration device 220 filters fluid from the collection device 210 before dispensing the fluid into an attached tube 212. Alternatively, the fluid can be collected in refillable or disposable bags for additional storage or purification. The filtration device 220 is a container including a filtration mechanism such as permanent or disposable filtration mechanism. The filtration mechanism can be contained in a hard or soft structure. It could be a unified system or a modular system. In some embodiments, the filtration mechanism is passive (e.g., a gravity-fed charcoal filter) with no power source. Alternatively, the filtration mechanism is an active filtration in which pressure is applied to the fluid via a powered mechanism (such as a pump) and forced through a filter, such as a screen or other implement capable of removing impurities from the fluid. The power source for active filtration can be any power source described herein.

The filtration device 220 can include supplementary passive components such as a charcoal filters or HEPA filtration systems or active components such as UV light sources or powered osmotic pumps. Additionally, the filtration device 220 can include ports for introducing pharmacological agents functioning to remove bacteria, fungus, protozoa, parasites, virus, or prions. These pharmacologic agents can be replaceable or refillable. The filtration device 220 can include a gauge to monitor functional parameters of the filtration device 220. Ionic filtration of removal of metals or other contaminates can be included to purify the water or other fluids. Filtration can be completely passive using gravity or can be power or partially powered. Different components of the filtration can be connected or able to be separated in order to control the exact filtration based on needs. Additionally, these components can be replaced as the capacity of each component is exceeded or exhausted.

The system 200 can include any two-way communication components as described herein. In some embodiments, the irrigation fluid collection and filtration system includes localization devices, such as GNSS devices. The system 200 includes memory to store identification information of the system and connected components for tracking and transmission. The system 200 can also record samples of contaminants filtered, such as ions, bacteria, parasites, metals or other contaminates. Sample filtrates can be collected and stored for evaluation and analysis at a later time.

In some embodiments, the system 200 includes one or more purification devices (e.g., decontamination devices, sterilization devices). Such devices can include light sources (e.g., UV light source 224), radiation, or gas devices. As a first example, UV light sources 224 arranged around a flow pathway or holding chamber 222, shown in FIG. 2C, purification fluids contained therein. The fluid drains into or pumps through such a holding chamber 222 where the fluid is maintained for a set time duration. Alternatively, the fluid is passed through the chamber 222 at a controlled rate of flow to ensure the fluid receives a sufficient purification dose, e.g., the fluid flows through the holding chamber 222 at a set rate and the UV dose rate is sufficient to purify (e.g., decontaminate, sterilize) the fluid. Examples include a specific length of tubing with a determined flow rate ensuring purification of a set volume within a set time. Alternatively, the fluid can be batch processed by filling the holding chamber 222 with fluid and treating with a set UV light dose to purify the fluid. In some embodiments, the holding chamber 222 is a drip chamber 223 of a fluid bag (e.g., IV bag) such as that shown in FIG. 2D. A UV light source 224 exposes the interior volume of the drip chamber 223 for a set dose thereby purifying the contents.

In some embodiments, the UV light source 224 is integrated into the holding chamber 222 or is temporarily attachable. For example, the holding chamber 222 can include a port, such as a twist-lock port, through which a UV light source 224 with a mating connection, such as the UV light source 225 of FIG. 2E, can be inserted and affixed and fluid within the holding chamber. In some embodiments, the UV light source 224 is a self-contained (e.g., battery powered) light source and housed to prevent fluid infiltration (e.g., water proof). In such embodiments, the UV light source 224 may be placed within the holding chamber for continuous purification. An example of this would be a reusable IV bag that has a twist cap on one end. A UV light stick can be inserted through the hole in the twist cap. The UV light could be attached to a cap that would twist onto the bag creating a seal. A metered dose of UV light would be used to purify the water. The water could be utilized via an alternate port without removing the UV light source and allowing for contamination from an unsterile top being replaced.

In some embodiments, the holding chamber 222 is a fluids container, such as an IV bag 228 of FIG. 2F, in fluid connection with the filtration device. The IV bag 228 can be exposed to a UV light source 224 for a time duration to achieve a dose thereby purifying the contents of IV bag 228. In some embodiments, the IV bag 228 is enclosed within a surrounding envelope. The envelope includes at least one transparent surface enabling an external UV light source 224 to purify the contents. In some embodiments, an envelope non-transparent surface facing the IV bag 228 is coated in a reflective material, reflective in the UV wavelength range (e.g., 100 nm to 400 nm). UV light reflecting from the coated surface re-exposes the contents of the IV bag 228, reducing the time duration until a purification dose is achieved.

Alternatively, a prefabricated sleeve or pouch could be designed inside an IV bag. This pouch, window or sleeve could be made of a different material that transmits UV light easily allowing for purification of the fluid within the bag without direct contact of the fluid with the light. In this array, the UV light source would fit into the sleeve and allow for purification, but the majority of the IV bag would still be fabricated of a material suitable for storage and use of fluids in an austere environment.

In other embodiments, a reusable IV bag may be used. The IV bag can have a threaded cap that would be able to be removed. A thin UV light transudative (Does not filter out or block UV light) sleeve is placed over the top of a UV light rod or dipstick. The UV light with the sleeve is placed inside the reusable bag. Once inside the IV bag, the UV light is activated to purify the filtered water. After treating the contents of the bag, the dipstick is removed but the now purified sleeve would remain in the bag. The cap would be screwed back on top of the bag. The concern for the cap being impure is obviated since the sleeve acts as a barrier between the purified water and the unpurified cap.

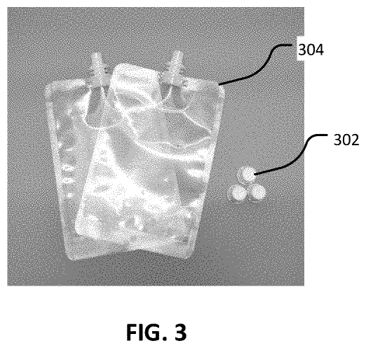

The purified fluid flows from the system 200 through a closed (e.g., tube) or open (e.g., vat) system into an irrigation platform, such as an IV bag. Irrigation platform access (e.g., a port or vent) allows for adjunct addition to the purification fluid, such as an adjunct fluid or dissolvable pharmacological agents. As shown in FIG. 3, for example, a dissolvable tablet 302 (such as NaCl or antibiotic powder) can be added to the irrigation platform 304 creating a specific irrigant composition to prevent or treat infected wounds in an acute setting. The tablet 302 or preformulated treatment is designed to be mixed with a predetermined volume in order to create a predetermined concentration of irrigation, such as 1 L or 500 mL.

Adjunct addition with specific intended effect (e.g., antibiotics, antiseptics, vitamins, or minerals) can be included in the irrigation platform 304 prior to their being filled with carrier fluid (e.g., purified water) or after they are filled. In one embodiment, the carrier fluid is purified. The carrier fluid passes into a sterile irrigation platform 304, via a sterile lock connector. For example, local potable or non-potable water is purified in the field and collected in a sterile irrigation platform 304 including tablet 302 of NaCl at a concentration that results in a standard IV fluid (e.g., 0.9% Saline).

This system can be utilized to provide drinking water as well for military personnel or in a mass casualty setting among other situations.

An alternative use includes a means to create normal saline for resuscitation in a trauma setting. Potable water can be converted to normal saline (NS) or sodium lactate solution (e.g., ringers lactate) based on the tablet 304 deposited into the preset volume of clean water created via the filtration system 200. A similar means of collecting exudate from a wound and spinning it down manually through a manual centrifuge to obtain packed red blood cells in order to resuscitate a patient or injured soldier. A wound interface component 120 could collect the drainage or bleeding from a wound and the collection canister (e.g., such as exudate canister 104) could be used to spin down the blood and auto-transfuse the injured person/patient. These blood products can be purified and resubmitted into the body in order to replace blood loss.

The device can include a small suction device allowing for use in remote locations such as camping, military zones, or disaster areas, where stable electrical power sources are unavailable.

The tubing at the end of the purification system can incorporate a backflow valve that does not allow retrograde flow of blood or bodily fluids so it can be reused with multiple patients. The system can be used to purify urine back to water. This could be useful in areas where water is not available such as in space.

The UV light purification system can be designed to be reused in reusable IV bags. The IV bag would have a cap that screws on to seal the bag. The bag can be filled with filtered water. Once the bag is filled with water, a reusable UV light wand can be inserted into the bag with a screw on collar that screws over top of the threads on the IV bag. This set up would allow for purification of the contents inside the bag and the inside wall of the bag with direct exposure to the UV light. However, the inside surface of the screw on top would still be unsterile.

In order to sterilize the cap, a portion of the UV wand that is inserted into the IV bag can extend outside of the bag. A second UV light would be used to sterilize the cap. The UV light that extends outside of the bag would have threads for the cap to screw down onto. The second UV light would then be positioned to sterilize the cap once the cap is screwed down to the top of the sterilization wand. A wire or button extending off the wand outside of the bad would be used to activate the UV light or other form of radiation or sterilization. A preset or controllable time frame can be used to insire sterilization or dosage of radiation. This dose would be used to ensure adequate sterilization.

Alternatively, a single use bag can be designed with premeasured NaCl inside the bag. Additionally the UV light could be built into the cap that is screwed on and activated with a light. The actually under surface of the cap could be the UV light. The cap would have an activation button as well as a small rechargeable battery for power.

The UV light could be built into other areas outside of the cap. Reflective material can be used inside the IV bag to magnify the UV light. A window to determine the amount of fluid inside the bag can be created for filling instructions and usage information. Lines can be created to allow for estimate of volume inside the bag.

The entire system or components of the system can be created in order to sterilize then system or bags or tubing in order to be reused. In a setting of mass casualty, natural disasters or military conflict, this system can be used to create IV fluids for different subjects. In a setting of high needs, everything down to the IV catheter can be reused in order to maximize the effects a limited amount/supply of resources. Cleansing can be accomplished through heat, solvents, UV lights or other means in order to reuse the components as much as possible to create the largest impact in a safe manner. Back flow valves, detachable components and refillable/reusable components can be utilized.

### D. Non-Electric Pump

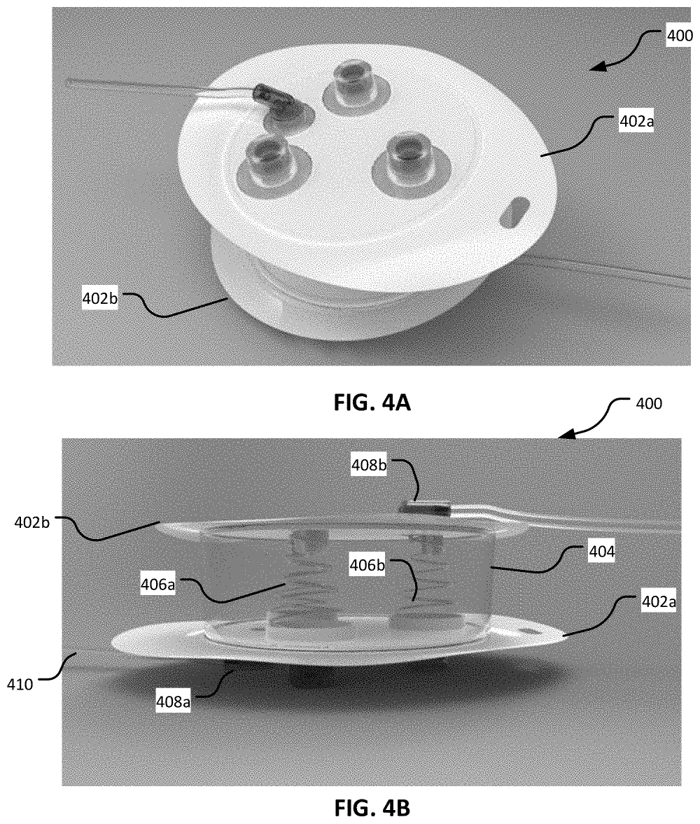

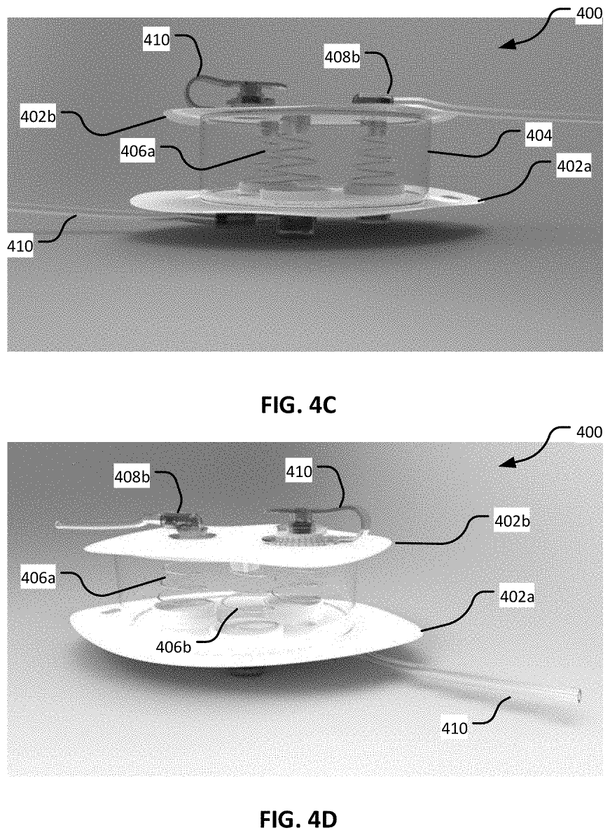

Referring now to FIGS. 4A-4D, the non-electric pump 400 including two end plates 402a, 402b and a spring-loaded collection canister 404. FIG. 4B depicts springs 406a, 406b within the collection canister 404. Alternative constructions may exist as well that utilize magnets or other means to promote negative gradients. The non-electric pump 400 is capable of prolonged use and creation of a vacuum pressure gradient that corresponds to the number, size, or spring constant of the springs 406a, 406b. Non-electric pump 400 can create a sub-therapeutic vacuum pressure gradient of between about −50 mmHg and about −125 mmHg. For example, in some embodiments the non-electric pump 400 can create a sub-therapeutic vacuum pressure gradient of about −60 mmHg. The pump or canister can have disposable bags in order to reuse the canister for multiple applications or patients.

The non-electric pump 400 includes two one-way valves 408a, 408b attached to respective end plates 402a, 402b of the collection canister 404. One of the one-way valves 408a receives inflow from the wound interface component while the other one-way valve 408b dispenses outflow (e.g., an in port 408a and an out port 408b). One-way valve 408a is arranged on first end of end plate 402a and attaches to suction tubing 410 coming from the wound interface component. One-way valve 408b is arranged on the opposite end of end plate 402b and dispenses exudate or irrigation fluid as the canister is pumped. Pumping by compressing the canister (e.g., compressing springs 406a, 406b by applying opposing forces to the end plates) evacuates the canister 404 interior volume and releasing the canister (e.g. allowing springs 406a, 406b to expand) can cause a vacuum pressure gradient applied to the wound interface component. Active pumping of the non-electric pump 400 enables active evacuation of the wound interface component and canister 404 in high flow events such as irrigation. In some embodiments, one way valves 408a, 408b function in the same flow direction.

The non-electric pump 400 can be pumped by foot or hand to create a vacuum pressure gradient applied to the wound interface component to remove exudate via tubing 410. Referring now to FIGS. 4C and 4D, in some embodiments, in addition to the two one-way valves 408a, 408b the non-electric pump 400 can include at least one pressure release valve, such as pressure release valve 410. FIG. 4D is a second perspective of FIG. 4C.

The non-electric pump 400 canister 404 is collapsible. End plates 402a, 402b are compressed together forcing any fluid in the interior volume of the canister 404 through the outlet one-way valve 408b thereby allowing pressure-assisted discharge fluid collection. The inlet one-way valve 408a prevents retrograde flow towards the wound interface component. Releasing end plates 402a, 402b directs the vacuum pressure within the canister to reinforce the wound interface component vacuum pressure gradient or remove any fluid in the wound interface component.

In some embodiments, a manual vacuum pressure gauge of the non-powered pump measures the vacuum pressure within the canister 404. A red or green zone on the display of the manual vacuum pressure gauge demonstrates the vacuum pressure gradient to be achieved. The outlet one-way valve could be attached to drain tubing exposed to the external environment in a trauma setting, or directed to a collection bag for collection and disposal.

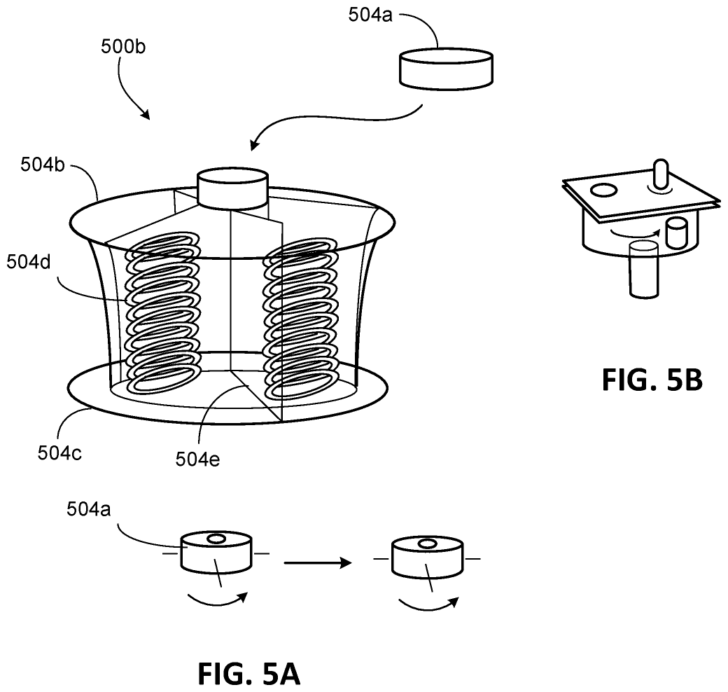

In some embodiments, an external powered pumping device functions as the pumping mechanism instead of a foot or hand. The external powered pumping device provides powered vacuum pumping with limited size and power requirements for home use or austere environments as in the military or during a commercial flight or military evacuation. FIG. 5A depicts powered pumping device 500a for use in combination with a non-powered pump 400. The non-powered pump 400 fits between the compression plates 502a, 502b of the powered pumping device 500 which moves compression plates 502a, 502b respectively toward the other to deliver pumping pressure.

The powered pumping device 500a can include a sensor housing 504 including one or more sensing devices for recording, measuring, controlling, or modulating the amount, rate, and application time of pressure. In some embodiments, the sensor housing 504 further includes a display for displaying information to the user. The external powered pumping device 500a can be powered by portable devices (e.g., solar, battery, mechanical cranks) or wired capabilities (e.g., plugged into a wall).

The powered pumping device 500a can operate in various modes depending on its use in providing wound therapy. In some instances, the powered pumping device 500a operates in an irrigation setting in which a chamber is compressed at time points that are separated by a specified time delay period (e.g., five seconds). In such instances, compression of the chamber can be used to produce a set flow rate within tubes connected to the compressible chamber. In other instances, the powered pump 500a operates in a maintenance setting in which the chamber is compressed to a specific height (e.g., 50% of the full height of the chamber when fully expanded). In such instances, the powered pump 500a operates similar to a mechanical pump that applies pressure to push down on top of the chamber and then releases the pressure applied to the chamber. Like the irrigation setting, compression can be repeatedly applied using a specified time delay.

Alternatively, the setting can be designed as maintenance. This setting would result in the powered pumping discharging or compressing the end plates once the end plates are separated by a certain distance or the negative pressure decreases past a certain threshold. This maintenance stetting would only engage or activate once a certain threshold is achieved in order to maximize power or battery life. Batteries can be rechargeable or solar powered in order to extend duration.

FIG. 5B depicts powered pumping device 500b for use in combination with a non-powered pump 400. In this example, a top portion of the non-powered pump 400 is attached to an attachment module 504a and fit between compression plates 504b and 504c. The powered pumping device 500b includes one or more compression cords 504d that radially extend from the attachment module 504a and terminate at a junction point 504e on a surface of the compression plate 504c.

Powered pumping device 500b can be used to compress a compression chamber by rotating the attachment module 504a relative to the compression plate 504b, which causes retraction of the one or more compression cables 504d and thereby reduces their length. Because the compression cables 504d are tethered to the junction point on the compression plate 504c, however, the shortening causes compression plates 504b and 504c to move closer to one another, which then results in compression of a compressible chamber. In some embodiments, the attachment module 504a can include a rotating motor that enables automatic retraction of the compression cables 504d. For example, the attachment module 504d can include a battery that provides power to the rotating motor.

Alternative modalities could use magnets, hydraulic presses, alternating directional springs.

The powered pumping device 500b can operate in various modes depending on its use in providing wound therapy. In some instances, the powered pumping device 500b operates in an irrigation setting in which a chamber is compressed at time points that are separated by a specified time delay period (e.g., five seconds). In such instances, compression of the chamber can be used to produce a set flow rate within tubes connected to the compressible chamber. In other instances, the powered pump 500b operates in a maintenance setting in which the chamber is compressed to a specific height (e.g., 50% of the full height of the chamber when fully expanded). In such instances, the powered pump 500b operates similar to a mechanical pump that applies pressure to push down on top of the chamber and then releases the pressure applied to the chamber. Like the irrigation setting, compression can be repeatedly applied using a specified time delay.

In the case of the low powered pump as well as the irrigation collection and purification system, each system can be stored in a small compact size in order to be placed in a medic pack. Each system can be interchangeable and work with different units. The modules can be exchanged or replaced in order to maintain system use.

The compression mechanisms can be built into the pump canister design or completely separate. In the example of a separate system, the powered compression can be designed to allow compression of the canister with endplates that slide over the canister. These plates may compress the endplates of the canister independent of the canister. In other words the canister would be low technical design and not have any built-in scaffolding for the motorized/powered pump to connect to. An alternate design would allow for the powered compression mechanism to be built into the canister design already. An example of this would be cords or string that would be placed at the center of the lower plate. Four (or more) cords would then wrap around the canister and meet in the center of the top plate. This design would split the canister into quarters in order to obtain even pressure on the canister and look similar to a ribbon on a wrapped present. The top of the end plate could have a winding mechanism built into the top end plate. The motorized or powered part would simply insert into the winding mechanism and apply preset winding actions when indicated. The small motorized unit would be detachable allowing for easy storage and transport as is needed in a medic backpack. Power sources can be rechargeable and/or solar powered, mechanical powered or powered via chemical reactions.

Backflow valves, universal connectors and component separation can be utilized in order to allow a single mechanical pump be used on different wounds and different injured people. Valves in the tubing can be used to close the system in order to maintain negative pressure between suction sessions. Back flow valves in the tubing can prevent biological contamination between subjects.

### E. Gravity-Independent Mechanical Wound Therapy Canister

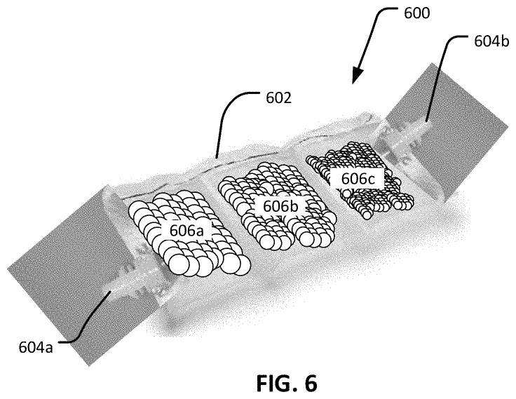

As shown in FIG. 6, the gravity independent mechanical wound therapy suction canister 600 can include a multi-chamber fluid bag 602, an entrance port 604a and an exit port 604b. The entrance port 604a can be configured for connection to a mechanical wound therapy device, such as a NPWT device. Fluid bag 602 includes three chambers 601a-c. Each chamber 601a-c is separated by a net or mesh. A suction line (e.g., fluid or gas) connects via tube to the entrance port 604a in fluid connection to the first chamber 601a of the canister 600. The entrance chamber 601a collects solid material prior to advancing down smaller flow pathways within the canister 600. The entrance chamber 601a can include a filter cage including holes or, alternatively, a solid wall requiring air flow to move through a 90 degree turn thereby dispersing solid material as the air flows around the turn. These pathways can be static pathways, such as one or more tubes and tube connections, or void spaces between objects, such as a bag of spheres 606a, 606b, and 606c such as those depicted in FIG. 6. The tubes and connections of the static pathways can include airway vents (e.g., vented tubing). Material surrounding vented tubing could be absorbent granules or sand to dehumidify gas as it passes through the vented tubes.

As shown in FIG. 6, the spheres 606a are larger than the spheres 606b, which is larger than spheres 606c. For example, the spheres 606a can be marble size, the spheres 606b can be pea size, and spheres 606c can be BB size. The spheres 606a-606b can be positioned in the fluid bag 602 in order of decreasing size or increasing size. For example, the spheres 606a can be positioned in the chamber 601a, the spheres 606b can be positioned in the chamber 601b, and the spheres 606c can be positioned in the chamber 601c such that fluid flowing into the entrance port 604a flows over the relatively large spheres 606a prior to flowing over the relatively medium sized spheres 606b, and then over the relatively small spheres 606c prior to exiting through the exit port 604a. The spheres 606a-606c can be hydrophilic such that as moist gas flows through the fluid bag 602, moisture from the moist gas collects on the spheres 606a-606c. These spheres can be expandable or constant in size. A benefit of not expanding would be to allow for continued flow as the spheres collect fluid. Expandable spheres would close off pathways for gas. Alternatively, cages or containment systems could be designed where the spheres cannot expand past a certain size in order to allow for maintained flow pathways.

The canister 600 contains between 250 mL and 1500 mL of total interior volume and is pressurized, e.g., not be dependent on gravity for fluid flow, allowing mobility for patients using the canister 600. In some embodiments, the bag 602 includes a carrying mechanism, such as a hook or strap to be worn on a belt or belt loop, or a strap or harness to be worn around the neck/shoulder of the patient. Alternatively, the bag could have built in ribs or structural supports that prevent the bag from collapsing. This scaffolding would maintain a minimum volume in order to allow for maintained flow pathways. The sphere arrangement by definition would maintain a minimum volume and allow for flow pathways assuming the spheres do not expand and close of the pathways. A combination of spheres or other geometric shapes (cones, stars, hexagons . . . ) as well as porous channels can be used to maximize flow and absorption.

Alternatively, the canister 600 can be a reversed: gas flows into an open chamber (such as 601a) with more than one partitions containing shaped absorbent material (such as that shown in FIG. 6B) of varying volumes. The flow pathway opens and the gas/fluid separates via a flow over or in between these partitions. The partitions containing absorbent material vary in shape and size and are maintained in their respective orientations via partition barriers, such as a mesh, or net, allowing gas and fluid to flow over the shaped absorbent material. The volume of shaped absorbent material ranges between 1 cm³ and 4 cm³ thereby collecting fluid and any foreign matter/particles, such as thickened clots or exudate.

The total volume of shaped absorbent materials in the canister 600 can include multiple shapes and volumes of individual absorbent materials and in some embodiments be arranged according to shape volume. For example, high volume (e.g., 5 cm³ or more) is partitioned at the distal end of the canister where absorbent material forming smaller shapes (e.g., shapes between 1 cm³ and 5 cm³) partitioned in the middle section. The section nearest the canister exit includes shaped absorbent materials with volumes below 1 cm³. The canister exit chamber is a high volume (e.g., greater than 50 mL) chamber including additional absorbent material. The exit chamber absorbent material absorbs fluid from gas flowing through the exit chamber thereby expanding until air flow pathway is prevented. Once the flow is prevented, a connected EVR system indicates an alarm notification indicating a full bag. Optionally, the connected EVR could include color-based flow indications depicting the canister saturation level.

In some embodiments, shaped absorbent materials and partitions allow expansion or, alternatively, do not allow expansion. Shaped absorbent material expansion limits flow as the material is saturated with absorbed fluid. The partitions can independently allow, or not allow, expansion. For example, larger partitions not allowing expansion, whereas the intermediate and smaller partitions allow expansion and thereby limiting gas flow as the shaped absorbent materials saturate.

These shaped absorbent materials include a solid surface or include a porous surface allowing multiple flow pathways thereby increasing surface area exposure to interstitial gases and fluids. In some embodiments, these pathways are constructed into the structure of the shaped absorbent materials with rigid components, such as wires or plastic frames, or constructed as static voids (e.g., holes) in the substrate.

These bags can have two way communication in order to sound and alarm once full and shut off the suction device such as an EVR. Communication can then be performed via text of voice to the patient or provider to initiate change of component. This can be documented in the medical record or offsite treatment facilities if the patient is in an extended care setting, home care or wound care facility setting in order to monitor compliance and treatment regimen as well as wound healing.

Alternative to a bag as described above, a metal or plastic frame can be designed that will allow for disposable suction bags to be applied to the frame. These disposable bags would fit over the top of a wire or plastic frame. The frame with the bag applied would allow the thin plastic bag to resist suction or negative pressure. This frame would allow for significantly less space and waste associated with standard canisters. That are large hard canisters. Disposable bags around frames similar to trash bags at events that are maintained using wire frames that are reusable, would allow for reduced waste, storage and cost.

Additionally, biological filtered or charcoal filters can be used to filter out non-fluid exudates to prevent clogging of the absorbent materials. One way valves can be utilized to prevent back flow and fluid management/compartmentalization.

### F. Tensioning-Bladder Combination Device

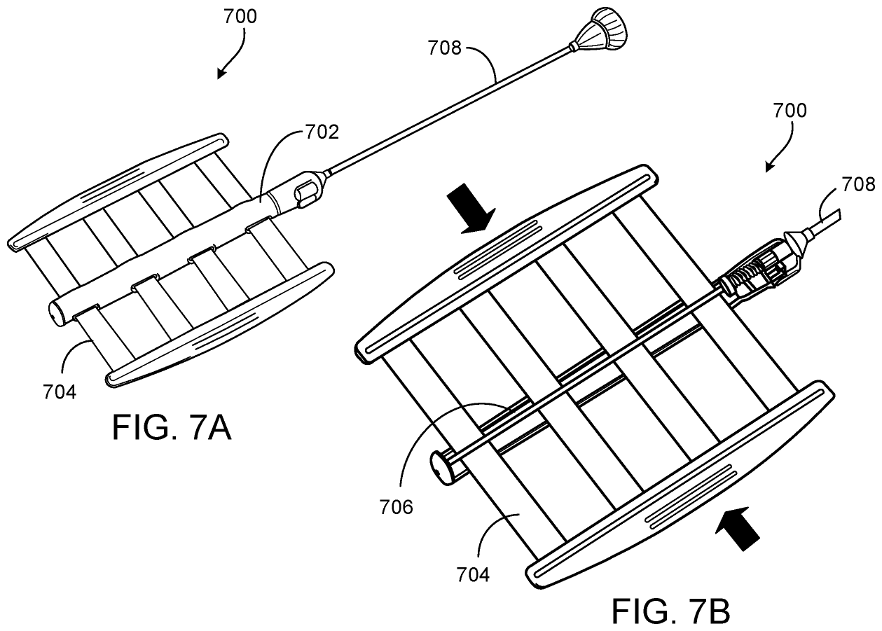

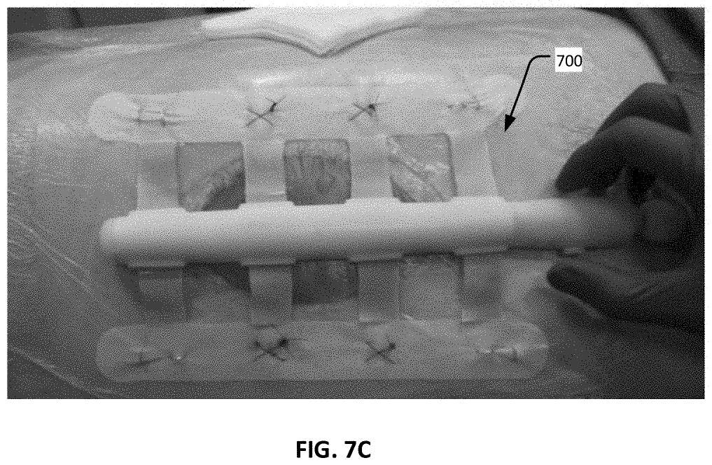

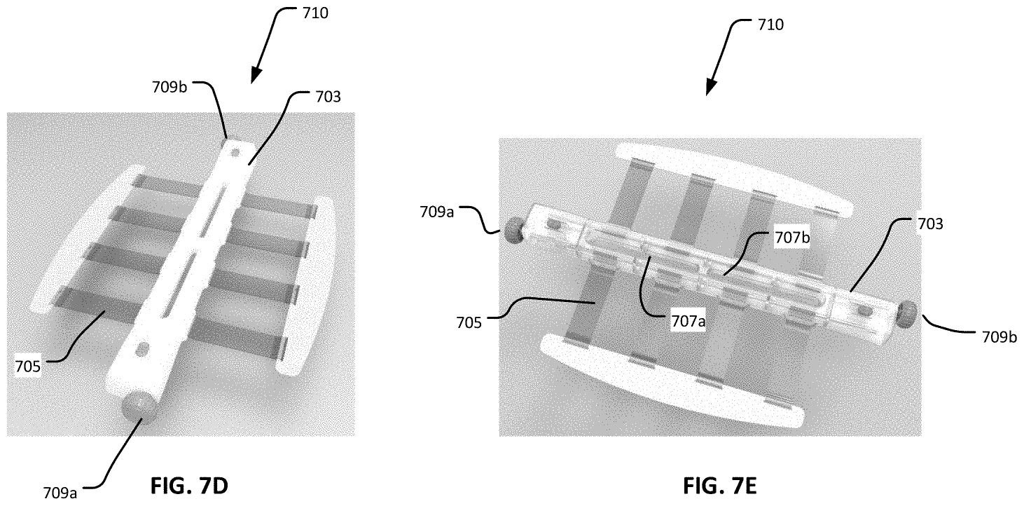

An example tensioner 700 (e.g., such as tensioner 116 in FIG. 1) for use in the system 100 is depicted in FIG. 7A. Tensioner 700 includes a housing 702 (e.g., a housing 702) with tensioning ribbons 704. As shown in the exploded view of FIG. 7B, the ribbons 704 connect to axel 706 which is rotationally operated by connected spindle 708 in FIG. 7A. Tensioner 700 operates in combination with a unidirectional bladder (not shown) attached between the tensioner 700 central housing 702 and the wound. This bladder inflates periodically to tension the ribbons 704 pulling wound edges together. During deflation, the tensioner 700 pulls the skin edges together. A tube connecting the bladder to a manual or electronically powered pump can be incorporated to allow periodic inflation/deflation. FIG. 7C depicts the tensioner 700 employed in a wound interface component on a patient. The tensioner 700 could be constructed in the form of a wire or plastic frame the rests on the wounds surface on top of the wound interface component. This frame could be sutured to the wound/skin. It could be attached via adhesives or simply use tension from the ribbons.

The pressure is manually or automatically controlled via a control mechanism, such as spindle 708, enabling control of the tension amount placed on the skin edges. Additionally, the time or duration of inflation, the speed of inflation and the duration of deflation can be controlled via separate control mechanisms.

FIGS. 7D and 7E show an example tensioner 710 with a dual coil mechanism. Two central coiling rods 709a and 709b can allow for eccentric placement of the housing 703 in order to visualize the wound easier. As shown in FIG. 7E, the housing 703 has windows 707a and 707b that permit visualization of the wound. With two coiling rods 709a and 709b, one side can be locked in a shorter position (i.e., ribbons 705 are only 1-2 cm extended). The contralateral side can be extended further (i.e., 10-20 cm). In this configuration the longer ribbon side would allow for visualization of the wound through the ribbons 705 with the housing 703 being offset to the shorter ribbon side. As the wound is tensioned the longer ribbon side is wound instead of the shorter side.

Alternatively, a single coiling rod can be utilized with the shorter ribbon side being static. A short static side will allow for the ribbons to expand unidirectionally placing the housing on the short ribbon side of the wound.

Two way communication can assist in maximizing the management. Tension or torque sensors can send feed back to the EVR which can transmit that data to providers. Optimal tension can be programed in order to increase tension or decrease tension based on the wound and patient desires, tolerance and conditions (such as swelling, infection . . . ). Control can be performed remotely or through the EVR. The EVR can control the tensioner with regards to tension settings, duration, sequential rate. NIRS or UV light and other powered modalities can be included at the wound surface of the housing in order to allow for additional monitoring or intervention. UV light can be used to detect bacterial counts in some instances. NIRS can ensure the tensioning is not too tight for two long resulting in tissue ischemia. This data can be recorded and communicated through the EVR to the system. The tensioner can have unique identification numbers for tracking and management of remote patients.

The tensioner 700 can include a limiter mechanism as a protective feature to prevent ischemia of the tissue on or under the skin. There will be a release mechanism to stop or reverse tensioning, for example, to examine the wound or for pain relief

The tensioner 700 can have identification information, two-way communications components, memory, storage, and other components as described herein.

The ribbons 704 of the tensioner 700 are substantially transparent and can be elastic or non-elastic. The ribbons 704 are composed of plastic or other types of material and formed into cords or ropes. In some embodiments, the ribbons 704 can be tape or suture wire. Ribbons 704 can be a sheet. The ribbons 704 can be woven material.

Ribbons 704 can be trimmed to match the dimensions of the wound. The ribbons 704 can be attached to the skin or wound edges via suture (as shown in FIG. 7C), staples or adhesive on the end of the strips. The length of the ribbons 704 can be cut short or longer in order to tension the wound with different widths along the axis 706.

Alternatively, the paddle that maintains the length of the ribbons and allows for ribbon control can be constructed in a manner to allow for easy detachment. In one configuration, the paddle can be sutured, adhered or stapled to the skin. The side facing away from the skin can have Velcro or other re-attachable means. The paddle part that is attached to the ribbons can have matching Velcro in order to allow for the ribbons to be easily released or removed from the skin edges to visualize the wound. The paddle part that faces the skin can have a slight adhesive that allows for easy placement without slipping or loss of position.

The ribbons and their attachment to the paddle can be static or adjustable. This would enable uniform tensioning in uneven or irregular wounds. In this configuration, the skin contact paddles would have a sticker baker that is removed and the paddles are placed on the skin outside of the wound on the periphery. The skin attachment paddles would be reinforced with staples or sutures to prevent skin tensioning and delamination of the epidermis. Next the tensioner would be expanded and the ribbons stretched out. The paddle attached to the ribbons would them be affixed to the skin paddle via Velcro. Once the two paddle sections are combined, the ribbons can be individually tensioned by pulling the ribbons through a channel or ratchet system for each ribbon on the paddle. The ratchet system could be similar to zip ties. A release mechanism can be devised to allow for release of tension when desired. After each individual ribbon is tensioned based on wound geometry, the entire system can be tensioned together using the central coiling rod(s).

The tensioner 700 housing 702 can be created in a flexible or compliant material in order to mimic the contour of the body its placed on. The housing 702 and components can be made see through or transparent. Alternatively, the housing 702 can be removed completely or be a wire or plastic frame to limit stiffness.

The tensioner 700 could be placed on an extremity such as a leg, thigh, forearm or upper arm. Alternatively, tensioner 700 can be placed over a torso such as the abdomen or back. The central housing 702 can be a single housing 702 or multiple housings 702.

As with the longitudinal tensioner, the ribbons can be tensioned at initiation individually with a ratchetting mechanism similar to pull ties. The individual arms can be tensioned at initiation or over the course of treatment. The ratchetting can be released as well.

Alternative configurations include a circular form in which ribbons 704 extend radially from a tensioning mechanism that twists like a screw to tension a circular wound instead of a linear wound. In this configuration, the ribbons 704 can be loops. The ribbons 704 attach to the tensioner 700 in a radial arrangement thereby allowing a circular wound to be tensioned in a uniformly radial (e.g., 360 degree) manner. The ribbons 704 are tensioned centrally via a twisting mechanism of the tensioner. Alternatively, the ribbons 704 could be pulled away from the wound dorsally.

The ribbons extend radially from the tensioner 700 and enter the tensioner 700 through channels. Within the tensioner 700, the ribbons wind around the central twisting mechanism. A series of these radial mechanisms can be designed to tension a linear wound with multiple round radial tensioners 700. These could be broken or cut into separate devices to use on multiple wounds or shorter wounds.

The tensioner 700 can include a NIRS sensor incorporated at the wound surface. This sensor could confirm appropriate perfusion under the tensioner 700 to insure there is no tissue ischemia due to over tensioning.

Windows in the tensioner can be created to allow for visualization of the wound. Alternatively, there can be pads that attach to the skin. The ribbons can be attached and removed from these pads that stick of are sutured or stapled to the skin outside of the wound away from the wound margin. The attachment can be via hooks, Velcro, latches or ridges that hook on a similar ridge.

The unidirectional bladder receives power from the EVR 102. Alternatively, the unidirectional bladder receives power from an external power source, such as a battery, solar power unit, or a wall outlet (e.g., AC/DC power). The unidirectional bladder can have communications components (wired or wireless) for communication with the EVR 102 or connection with a local or remote network.

The unidirectional bladder operates independently from or in conjunction with the tensioner 700. The bladder can be programmed to activate during irrigation thereby assisting in pumping a fluid to the wound surface increasing fluid return as well as improving clearance of exudate or wound debris. Unidirectional bladder activation during irrigation, particularly in conjunction with reverse pulse lavage, improves wound cleaning, reduces dead space, and increases wound interface component 120 movement on the wound surface preventing tissue ingrowth. Additionally unidirectional bladder fluid pumping can improve wound coverage during irrigation. The unidirectional bladder pumping mechanism decreases soft tissue edema, similar to a sequential compression device used to prevent venous congestion. The unidirectional bladder pumping mechanism improves wound coverage and delivery of medical (chemical or biological) agents to the wound surface, including delivery into sinus or cavity wounds.

The bladder can be inflated via its own pump or tubing can be attached to an external pump. That pump can be attached to the EVR for regulated inflation/deflation or it can be attached to a mechanical hand powered bulb pump as seen in typical manual bloop pressure measurement devices (sphygmomanometer).

The EVR 102 system can include more than one unidirectional bladder. For example, two unidirectional bladders on the wound interface component periphery or tensioner 700, and one in the centrally of the wound interface component. In such embodiments, the peripheral unidirectional bladder (e.g., wound interface component periphery) inflates to drive the fluid towards the central suction chamber. The central unidirectional bladder then inflates driving the fluid out of the wound interface component. The peripheral unidirectional bladder remains inflated during operation of the central unidirectional bladder to promote the removal of fluid.

The bladder can be designed to allow expansion in predesigned directions. A 3 leaf clover shape can be designed where the central leaf is directed downward to put pressure on the wound. The two side leaves can be directed in a lateral direction to allow pumping and tension on the lateral edges of the wound.

In a similar fashion, the unidirectional bladder can form a donut shape incorporating a second unidirectional bladder to pump fluid towards the central suction chamber. For example the peripheral unidirectional bladder remains deflated and the central bladder inflates with the peripheral part inflated and maintained inflated to pump fluid out of the wound interface component.

If pH changes indicating possible infection development occur, communication between the EVR 102 and wound interface component enable components, such as UV-C lights, to reduce bioburden in a controlled fashion. The unified construction wound interface component allows for connections to be integrated into wound interface component construction.

The tensioner can be used to stop hemorrhage in a battlefield or military conflict or in a mass casualty setting. In this setting the tensioner combined with a manual inflation device would allow for direct pressure to be placed on a wound similar to another person placing direct pressure on the wound.

When a wound is created, the tensioner would be placed over the wound. The ribbons would be pulled over the wound and the paddles stapled to the skin edges. The tensioner would then be tensioned to a higher tension than would be allowed in a non-traumatic setting. The torque release mechanism would be set at a high threshold as the purpose would be to place significant tension on the wound and underlying tissue in order to stop bleeding. Once the tensioner is tightened, a mechanical hand pump would be used to inflate the bladder. In this case the bladder can be similar to the unidirectional bladder, but it could also be a more stout material similar to the dorsal material in the previously described unidirectional bladder. In a trauma setting, the need to avoid puncture or popping due to higher pressures may prevent the use of the thinner elastic material that would dissipate the pressure placed on the wound. The bladder would be pumped up under the taught tensioner to mimic manual pressure on the wound.

In a similar fashion, the circular tensioner could have a bladder placed under it. Similarly, the circular tensioner could be placed on a circular wound. It could be tensioned as well and a bladder inflated under it to allow for more point pressure versus the more linear pressure of the central housing design. In both cases the mechanical pump similar to a sphygmomanometer bulb pump can be attached via tubing. This can be detached for storage. Any type of manual pump could be utilized for inflation.

The tensioner can be used in wounds that have been closed but are tight. When wounds are closer but the closure is tight, skin necrosis can occur at the wound edge due to the suture pulling too hard on the skin. The tensioner can be placed over a closed wound that can offload the skin edge at the wound. The ribbons can pull on the skin in a direction towards the wound to offload the wound.

Skin and wound perfusion in order to prevent over tensioning can be utilized. These modalities can be NIRS, pH monitoring, temperature monitoring, tissue probes or other means can be used to determine tissue perfusion. If indicators show poor perfusion, the tensioning can be released in order to allow for improved perfusion. Alternating between tension and non-tensioned setting allows for maintenance of adequate perfusion over an extended period of time. Biological feedback can be used to control frequency and duration of tensioning in order to maximize healing. Patient feedback such as pain can be utilized to prevent discomfort. Additionally, local anesthetics such as lidocaine can be used to alleviate pain and discomfort. Local anesthetics such as pain pumps or infusion can be used around local skin in order to limit pain and allow increased but safe tension on the wound edges.

### G. Barrier Device

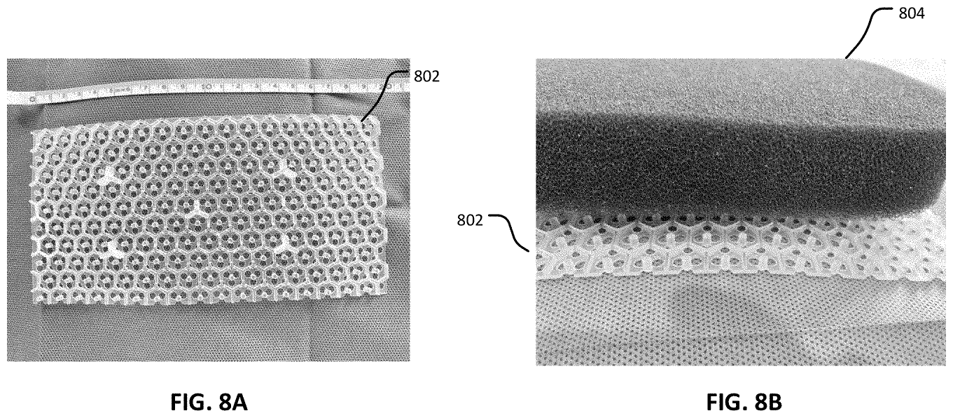

The device 100 includes a non-compressible scaffolding, shown in FIG. 8A as barrier 802, functioning as a barrier 802 to separate a sponge from the wound. The honeycomb structure of barrier 802 allows for tangential flow through sponge 804, shown in FIG. 8B. The honeycomb walls have holes or flow pathways that allow flow parallel to the wound surface. Vertical flow occurs through the perforated holes at the wound surface contact side. The barrier 802 can be unidirectional or bidirectional. The barrier 802 height can be between 1 mm and 5 mm. The non-compressible barrier 802 resists compression preventing contact between the sponge and wound. In some embodiments, the barrier replaces the sponge and operates as a wound filler. The barrier 802 is composed of a low durometer material (e.g., soft) and to mirror the surface of an uneven wound. The barrier 802 prevents tissue ingrowth and transduces applied suction across the entire wound. The barrier 802 can be made of ingrowth-resistant materials such as TPE, TPU, silicone, polymer, or plastic. A hydrocolloid or other adhesive can be used in order to extend the wear duration barrier 802 from a standard 2-3 days to 6 or more days.

When the barrier structure is used as a wound filler, the construct allows for a non-compressible structure or scaffolding that has a 3 dimensional shape that maintains flow pathways in both vertical and horizontal direction. This scaffolding maintains flow pathway and prevents wound tissue in-growth. It can be see through or transparent to allow wound visualization without wound interface component removal.

Irrigation or fluid/gas pathway can be incorporated into the barrier to allow for medication delivery into the wound. These barriers can be layered to allow for additional depth, The barrier is a closed cell that prevents material from being left in the wound similar to a sponge or woven fabric. The scaffolding can provide some compression in order to allow for pressure release or padding. The compression that is allowed or experienced would not allow for collapse of the flow pathways or holes in the honeycomb structure. The scaffolding can be designed in multiple geometric shapes such as circles, hexagons, triangles, stars or other shapes.

Alternatively an array of bumps or columns can be designed with similar or different heights that create a barrier or separation for the sponge or sealing layer form the wound. These series of side-by-side columns can be connected on a perforated sheet or other means. The columns can vary in length in order to allow for flow pathways.

The barrier or wound contact layer can be altered in order to allow for more compression to protect against pressure injuries. The durometer of the barrier can be modified or the structural design can be modified in order to allow for more compression/cushioning of the barrier. The less material or higher height can allow for more protection from pressure.

The barrier can be placed over intact skin prior to full thickness wounds in instances such as pressure ulcers. A light adhesive can be placed on the wound contact surface in order to place the barrier over prominent body parts prone to pressure ulcers. In this application, a sticker backing would be peeled off and the pliable barrier would be placed on the sacrum or the back of the heel. The barrier is soft and would allow for some offloading of pressure without complete collapse of the structure. The barrier could be made of a more compliant material to allow for more cushioning. The negative pressure could still be applied to intact skin and early stage pressure ulcers to promote blood flow and healing prior to ulcer formation. This management could be prophylactic to prevent ulcers using NPWT. The barrier and sealing layer can be translucent in order to monitor the skin and ensure it is still intact and an ulcer has not formed.

Additional tabs or circles that are slightly elevated above the dorsal aspect of the barrier can be designed to allow for dome or suction manifold placement. The adhesive layer needs to be pulled away from the barrier to allow for cutting of the sealing layer for manifold function. Structural elevations or depressions can be designed to facilitate the manifold application.

The dome can be designed to control 2 or more flow pathways. These pathways can allow for irrigation, medication delivery, stagnation prevention, or other purposes. The dome can be designed to maintain separation of the systems such that a wall can separate the suction aspect from the irrigation aspect or even a bleeder valve or stagnation/dead space prevention system. This pressure release area would be separate from the suction system so flow would not go through the suction tubing but instead would travel through the entire system and allow flow over the wound to facilitate fluid removal and prevent stagnation of a closed/sealed system. This system would have the bleeder tubing connected to a series of tubes or pathways that extends over the wound. So the release valve would allow air into the system through a filter or filtration system, this air would travel through and array of pathways that open to the wound surface over an extend area away from the central suction chamber. There for the air form the release vale would travel over the wound and increase fluid removal prior to be suctioned out at the central suction chamber.

The dome or connection to the system for the suction source can have 3 or more chambers. 1—A suction port that allows suction and removal of exudate or irrigation. 2—a filter that prevent dead space or stagnation. This filter can be capped or flossed off as well in order to induce stagnation or more commonly known as instillation of medications. If the bleeder valve is closed, then stagnation or pooling will occur even if suction is running. Alternatively, suction can be stopped or paused in order to allow for medications to be pooled on top of the wound. 3—the third chamber can consist of irrigation or in flow pathways. These pathways could terminate at the periphery of the dressing or wound or it could be a branched pattern that terminate throughout the wound surface. Either configuration may have advantages in different settings or treatment options.

The barrier can be coated with a single or multiple chemicals or medications in order to act on the wound surface. These medications can be delivered over a series of time intervals based on layering. The outer layer would be released first as it is activated or dissolved, The next layer then would be released and similar phases of release as time or activating/dissolving agents are used to release the medication or chemical. These agents would be designed to be released over time as the wound matures.

Pain relief can be used for example as a medication, or antibiotics or biologics or growth factors. Wound beds can be a means of providing medical delivery. Sublingual delivery is used as well as per rectum due to the vascular supply in these areas. The wound itself can be used due to the exposed vasculature to deliver systemic medications using the dressing to delivery the medications. Systemic absorption can be controlled and sustained levels of therapeutic chemicals can be achieved through episodic delivery or dwell times or sustained release gels/powders or coatings. Liposomal or designer chemicals can use utilized to adhere to the wound surface and be absorbed over time with delayed release agents,

Coatings can be activated or released based on activators and chemical reactions such as water or other washes that are delivered through the dressing or wound contact layer and its irrigation routes without exposing the wound to the environment.

The barrier or contact layer can have built in irrigation pathways or flow pathways to distribute the irrigation/medication/therapy evenly over the wound surface. Additional tubing or mechanisms may exist that allow for specific access to cavity lesions, tunneling wounds such as gunshot wounds or even cavities such as the abdominal cavity, the plural cavity, thoracic cavity or dural space. Multiple systems of branching pathways can exist and be separated. For example one system can be an inflow while the other could be out flow. Alternatively, there could be two in flow pathways that allow chemicals to be mixed or react at the wound surface but be delivered separately in order to allow for separation until at the wound surface. For example a clotting or hemostasis type thrombin or other chemicals can be injected through the system to allow for bleeding control. These chemicals may intact when mixed so delivery would require separation of the reagents until they are on the wound surface. One flow system may be used to limit or eliminate dead space or stagnation. Filters that limit flow and clean the air can be placed into the pathway system. Gases can be used such as oxygen or carbon monoxide or other gases can be used in therapeutic ranges to promote healing.

Positive pressure can be utilized through the irrigation system in order to prevent stagnation and promote exudate removal. Alternating positive and negative pressure can be utilized in specific sequences to promote wound healing. Alternating the direction of flow in the two or more systems can assist in preventing dead spaces or stagnation.

The opening of these pathways can be at a central port or hub. The access points can be on the periphery or in the tubing. Single or multiple ports can exist. Stopper caps or removable seals can be utilized to control flow on and off. Hepa filters or other filters can be used to clean the chemical, gas or liquid that is distributed to the wound.

These pathways can have valves similar to veins in the human body. These valves can be placed throughout the system or at the central suction port or other locations. These valves or simply thin material extensions within the flow pathways can act as one-way valves to prevent back flow. This design can assist in fluid removal without the need for high powered vacuums or suction. In a similar way to the venous system in the human body, a low-pressure system (venous system) still allows for return of blood through the actions of the muscles squeezing fluid towards the heart. In a similar fashion, the patient's movements, body weight as well as the tension and bladder combination described here can act as muscle and drive or pump fluid through the pathways through the use of one-way valves. These valves would prevent back flow or reverse flow and move the fluid or exudate towards a central suction chamber.

Conversely, suction performed through the radial irrigation tubing can allow for removal of fluids at the periphery of the wound that may erode the seal. The EVR can periodically reverse flow and suck through the inflow system to prevent clogging and allow for removal of stagnant material or debris.

The shape of the barrier or any dressing or wound contact layer can be designed specifically for deep or cavitary wounds. In this scenario the dressing would have wedges that are removed from the periphery of the barrier. This design would allow the barrier to lie flat against a cavitary wound without wrinkling. The design would create wedges that are removed with the wider end on the periphery and the thinner point towards the center. This would allow for a similar phenomenon to a coffee filter in a coffee pot. Wrinkles instead of wedges being removed are used to create a cavitary structure. Wrinkles or soft spots in the wound contact layer could be designed to allow for improved coverage in cavitary wounds.

A thinner version of the barrier can be created for more chronic wounds. The initial design has larger holes ˜3 mm of diameter and honeycomb walls ˜3 mm of height. Alternative designs can be made that have much thinner designs (˜2-3 mm) total height. The perforated holes can be much smaller and the honeycomb walls can be 1-2 mm in height. This design can be for lower flow wounds that are chronic in nature.

Additionally, larger designs can be created that have specific stiffness or lack of stiffness that allows for a offloading of pressure in areas that are prone to pressure injuries such as the sacrum, posterior heel. The perforated hole surface can either come with a sticky or adhesive material pre attached or a adhesive spray can be used to attach the cushion device to the skin to prevent removal of displacement.

The barrier can be used in several manners. 1—it can be used as a barrier to prevent in growth under standard non suction dressings. It can be used under a negative pressure dressing that allows flow and prevents ingrowth. 2—It can be used as a wound filler. The wound filler can be used with instillation with a NPWT dressing. 3—It can be attached to an adhesive cover to be a unified dressing. 4—tubing or venting or irrigation pathways can be created to allow for venting, or medication delivery similar to the unified dressing design. The dome or suction port can have any combination of suction, venting (filtered or non-filters, with controllable rates of flow) as well as an inflow system for medication or fluid/gas delivery. The inflow and venting can be closed off or capped to prevent flow in order to allow for dwelling of medication while maintaining continual suction. Controlled stagnation can be utilized to allow for dwell time of medication or therapies.

Two separate interdigitated flow pathways can be derived in the barrier design. One pathway system similar to the veins in a leaf can be designed to allow venting. A second and separate system that is interdigitated within the whole dressing or part of the dressing can be designed to allow for flow of gas/fluids/medications. Alternatively, if a dressing or barrier is considered to be a map. Two pathway systems can be designed where one system is directed East while the other is directed West. In this manner, fluids or medication is directed East and sucked across the wound towards the West suction end or vice versa. Additionally, a North/South set up could be designed. This two irrigation or delivery/suction pathways could be used to maintain two different reagents apart form each other until they are mixed at the wound surface allowing for a predisposed or planned reaction to occur at the wound surface. These chemical reactions allow for reagents or by products to be deposited at the wound surface in a global manner if the pathways are interdigitated.

The barrier or the unified therapeutic delivery system can be soaked, coated or have medications impregnated into the material. The coating can be activated or react to gases or fluids that can be delivered to the wound surface. These coating can be biological inert or active materials. It can be a cellular coating such as stem cells or proteins or other biologically active enzymes.

A leash or tab can be placed in or through the barrier or the UTDS in order to insure no piece is left behind. In some cases, the person who places the dressing is not the same person who removes the dressing. If a barrier or other dressing is placed in the wound, but it is not attached to the other parts of the dressing, a leash or tab can be placed through the holes in the dressing. This leash can have conspicuous characteristics that will draw attention to the dressing piece. It can be colored in a non-biologic color such as blue, green, neon. . . . These leashes or tab can have a long tail that can be trimmed or placed in the opening of the wound in order to draw attention to it in order to follow the tail down to the dressing or wound filler.

### H. Unified Medication Delivery System

Referring again to FIG. 1, the system 100 includes a wound interface component 120 including a central suction chamber and inlet valve 121 which attaches to inflow tubing. This component can be a part of a larger systemic system. The inlet valve 121 additionally can include injection ports through which fluids can be added to the wound surface through the wound interface component without passing through inflow tubing. This inlet valve 121 facilitates the addition of biologics, gels, or other therapeutics in order to promote healing. The inlet valve 121 allows back flow of fluid to enable, for example, clogs to be dislodged or the wound environment sampled. The inflow and outflow tubing can additionally include one or more ports allowing positive or negative flow from the outflow system. The wound interface component 120 can further include a two-way valve including a port that is exposed to the environment, such as a bleeder valve or release valve. The two-way valve is operable to expose a wound to an environmental gas (e.g., air) to balance the pressure at the wound with the environmental pressure. In some embodiments, the two-way valve further includes a filter. The two-way valve can be integrated with the inlet valve 121 or separate.

In some embodiments, the central suction area includes one or more light sources, such as fiber optic cables delivering light from an external emission source or low voltage LED lights, such that the central suction area is exposed to therapeutic light (e.g., UV light). The light sources can be lined in series along irrigation tubes connecting to the wound interface component. Alternatively, the light sources embed in the hydrocolloid sealing layer thereby providing light therapy to the wound. For example, the light or fiber optic cable can be embedded in a radial fashion around the central axis of the NWPT wound interface component 120. The radial alignment allows cutting peripheral portions of the sealing layer to match wound contours, without interrupting light transmittance.

In some embodiments, the NWPT wound interface component 120 includes components to produce Weak Electrical fields (WEF) therapy. Ion gradients, such as Ag, Zn or other ions, creates a WEF aiding treatment of infections. These fields can be powered independently via an internal or external power source, such as any device described herein, or dependently with the NWPT wound interface component 120.

The NWPT wound interface component 120 includes an identification information (e.g., serial number) to enable individual wound interface component and logging of components within the EVR system. The identification information is preset and additional identification information can be stored in memory including information corresponding to patient identification numbers, names, or locations, or identification information of components of the system (e.g., EVR, Pump, canisters, tensioner, hospital/facility monitoring system or remote monitoring system). The NWPT wound interface component 120 includes one or more sensors to monitor temperature, heart rate, pH, blood pressure, or perfusion (e.g., a near-infrared spectroscopy sensor). Changes in pH can indicate the development of a dead space or an infection.

The NWPT wound interface component 120 can include wired or wireless communication components thereby enabling two-way communication between the wound interface component and the EVR system and/or other command centers. The NWPT wound interface component 120 detects pressure gradients to detect leaks including localization information. The NWPT wound interface component 120 includes memory to store recorded data or transmit the data to connected systems.

1. Suture Wound Interface Component

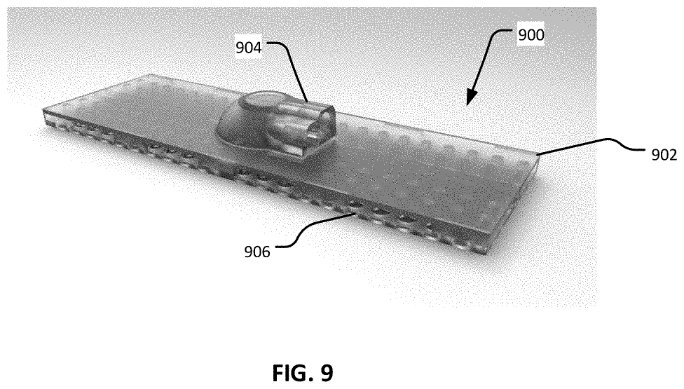

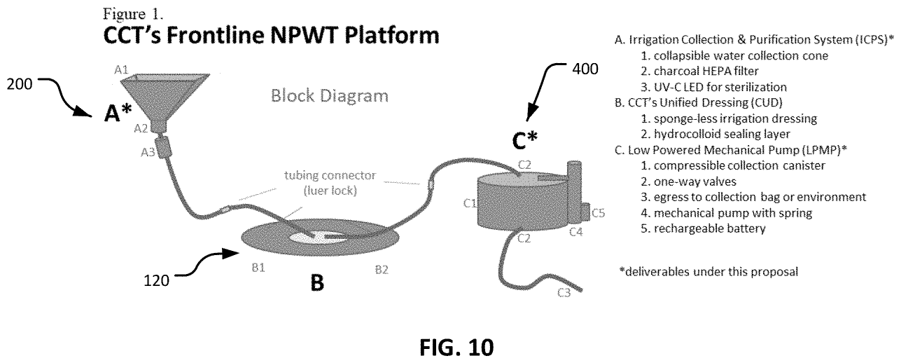

The wound interface component 120 of the system 100 can be a suture wound interface component 900, as shown in FIG. 9. Wound interface component 900 can be layered, allowing a smaller central suction chamber 902 due to low expected volume and limited irrigation needs. As shown in FIG. 9, this smaller central chamber 902 can be longitudinal instead of round as the need for suction will substantially be along the longitudinal direction rather than 360 degrees in round designs. By creating a two directional linear suction chamber 902, the chamber 902 can become narrower, further reducing the foot print. A narrow chamber 902 allows for much thinner connections 904 for the sealing layer and a thinner wound interface component 900 such that it only covers the sutured wound by between 1 cm and 5 cm in any dimension. Irrigation can include antibiotics or gases. The wound interface component 900 facilitates the use of both therapeutic gases and fluids to optionally dry or hydrate the wound. The wound interface component 900 can include filters to clean, dry, or nebulize irrigation gases.

2. Skin Grafting Wound Interface Assembly

In some embodiments, the wound interface component 120 acts as an allograft, or autograft, skin grafting mechanism. An allograft skin replacement can be already pre-affixed to the wound interface component 120. Integral or animal substitutes can be pre attached to the wound interface component 120 during manufacturing to allow for placement on open wounds. The wound contact layer on the wound interface component 120 can be modified to allow for more or less holes in order to maximize skin graft take. The means to fix the allograft to the wound interface component 120 utilizes spot welds to the perforated wound contact layer (e.g., barrier 802). Therapeutics such as collagen, allograft, autograft, amniotic patches or other means can be attached and delivered to the wound surface via these means.

The wound contact layer could be modified to be smooth, e.g., without perforations. The wound contact layer can also include longitudinal slots to allow suction or irrigation. A dissolvable fixation system can be utilized that dissolves when in contact with water, irrigant, or normal skin exudate. A biological adhesive can be used and be designed to degrade over time through time sequence or water dissolvable or other means such as enzymes that can be delivered through the wound interface component 120 irrigation system to free the wound interface component 120 from the allograft. Additionally, the wound interface component 120s and managements can be used in burn treatments.

The wound interface component 120 can include split thickness or full thickness autografts including any dissolvable medications, adhesives, or therapeutics as described herein can be coated on the bottom of the wound contact layer.

The wound interface component 120 allows biologics, such as amniotic tissue or other human tissues, stem cells or platelet rich plasma from the host, to be injected into the wound. These biologics can be pre manufactured or placed under the wound interface component 120 on the wound. The wound interface component 120 can be precoated with therapeutics or pharmacologic material that dissolves over time in order to manage the wounds. These materials can dissolve as they are exposed to water in order to release the chemicals for treatment of the wound. Different chemicals, such as antibiotics, biologics, stem cells, growth factors, can be bound to the wound interface component 120 so that the wound is exposed to these chemicals in a set time period or order in order to allow tailored wound management.

The wound contact layer of the wound interface component can be constructed completely of a dissolvable or biological material such as collagen. This layer can become part of the host as the wound heals in. This layer would be designed to encourage wound tissue ingrowth and vascularization. It can contain growth factors that encourage wound healing.

These wound interface components 120 can be created to specifically treat different types of wounds such as acute wounds versus chronic wounds versus peripheral vascular wounds. Based on the type of wound, the wound interface component 120 can be specifically designed to treat wounds such as treated with antibiotics for infected wounds, or with medications that increase vascularity for peripheral vascular disease wounds.

As a further example, a padded wound interface component 120 could be applied to the pressure ulcer wound therefore combining both chemical and physical design modifications of the wound interface component 120 for the needs of the wound and patient. The wounds can be characterized as, but not limited to, acute, chronic, dysvascular, diabetic, pressure ulcer or infected. This design would allow for specific tailoring of the wound interface component 120 to the type of wound from a pharmacological aspect.

3. Unified Wound Interface Component Hydrocolloid

The wound interface component 120 can include a hydrocolloid layer, replacing the sponge 804 of FIG. 8. Hydrocolloid wound interface components 120 are a unique type of bandage that provides a moist and insulating healing environment for wounds. The hydrocolloid can be a means to deliver therapeutics, such as biologics, to the wound surface or periphery. The hydrocolloid can join with wound-specific wound interface components 120. Wound interface components 120 coated with specific therapeutics, such as pharmacologics, can be operable with specific hydrocolloids formulated with specific pharmacologics that aid wound management and therapy. For example, hydrocolloids formulated with antibiotics can be used for infected or contaminated wounds such as wound with abraded or macerated skin e.g., rubbed off due to asphalt or cement seen in automobile accidents or off-road vehicles. Additionally, vasodilators, gases (e.g., oxygen, or nitrous oxide), anti-inflammatories, or vascular promoters (vasogenesis agents or growth factors) can be embedded in the hydrocolloid and released over time to the skin and periwound.

The hydrocolloid or adhesive can be impregnated with any different types of medications or therapeutics. Time released sequences can be designed to sequentially release medications in a timed sequence in order to allow therapeutic management over a specified time. This includes specific releasing molecules for gases or other agents that have different half-lives. This can be embodied as different dissolving rates for fillers or delivery systems. Different bonding rates can be utilized. Additionally the irrigation fluid can assist in releasing medication in the hydrocolloid. By irrigating the wound and contact interface with specific chemicals, that chemical could release specific preloaded medications within the hydrocolloid itself. Activating agents can release different medications (antibiotics, anti-inflammatory agents, growth factors) via use of different activating agents.

4. Reinforced Rebar

The perforated barrier 802 in the wound interface component 120 can include woven metal or suture to increase wound interface component tear resistance. For example, nylon sutures or thin metal wires added to the wound interface component 120 material during production (e.g., injection molding) to increase strength and prevent tearing of the wound interface component 120.

5. Biologics

The unified wound interface component 120 can be a means to deliver biologics, such as amniotic tissue, stem cells, platelet rich plasm (PRP), or other therapeutics and delivered to the wound over a continual basis or bolus means. The wound exudate, such as PRP, could be spun down, filtered, and recycled over the wound. Biologics such as amniotic fluid can be used to bath the wound. Therapeutics such as medicinal medications, herbs, or elements can be added to the wound. These therapeutics can be delivered through the wound interface component 120 or the wound interface component 120 can be coated with these medications which then dissolve over the course of the wound interface component 120.

The wound and entrained biologics can be sealed with a sealant and the therapeutic placed through the wound interface component 120 at the time of initial wound irrigation and debridement. Once the wound is cleaned the wound interface component 120 is placed and the wound treated with the entrained therapeutic. The wound interface component 120 sealant, or curing agent, creates a suction resistant biofilm or wound cap. Examples of sealant or curing agent include fibrin glue, hyaluronic acid, or thrombin gel. The sealant can be mixed with a biologic or therapeutic, or placed on top. The sealant protects the therapeutic from being removed during suction of the wound surface with mechanical wound therapy. The sealant is non-reactive to plastics, TPE, or other silicone or wound interface component 120 materials. In some embodiments, the sealant is colored to ensure full wound coverage, or reapplication indicator. Color coordinated managements can be devised in order to tailor the wound treatment based on type of wound, patient or chronicity of the wound.

A wound sealer can be designed. This wound sealer can be applied through the contact interface similar to irrigation or other therapeutics. Once the wound sealer is confirmed to be over the entire wound, an activator can be applied that results in a congealing or curing process. This could involve collagen or other biological scaffoldings. It could adhere to biological tissue but not to TPE or other resins that the tubing and system would be made of. Once activated the seal could cover the wound and protect it as it heals limiting infections and other detrimental occurrences.

Alternatively, a biofilm design could be performed where a chemical or mixture of chemicals is injected into the wound contact interface. After a specific time is allowed for the biofilm to cure or harden/establish itself, then standard irrigation or other medication delivery could be initiated

6 Cavitary Design

The design can allow for weak spaces to allow folding or have wedges cut out to allow for easier coverage of a deep wound without wrinkling. This would allow for easier wound coverage so the edges do not wrinkle when placed in a deep or bowl shaped wound versus a flat or shallow wound. The edges can still be trimmed as needed. Alternatively, the contact layer can be designed and molded as a concave or bowl-shaped system that allows for placement in a deep space.

7 Daisy Chain Design.

The dressing can have a single suction tubing that connect multiple either unified dressiness or prevents. These multiple dressing could be in series or in parallel. The dressing could be used as needed. If an injury consisted of multiple wounds such as 3 wounds and the daisy chain had 5 branches with 5 separate dressings, then two could be removed. A system would be in place where removal of an unneeded dressing would not result in an open leak for suction or irrigation. A pre-designed closure would be used or he tubing could be tied or clamped to prevent a loss of suction. This system would allow for management of multiple wounds or multiple areas of complex woulds such as limb amputations in blast injuries seen in military conflicts. A single suction unit could service all the different wound or management areas. Additionally, irrigation and medical delivery could be performed throughout the wound(s).

These separate dressings can be termed leaf dressings with a single branch type design to allow suction and inflow. These branches can be cut if not needed or clamped to put the leaf out of commission.

An example could also involve and inflatable ring around the central suction chamber. A mechanical pump can be used to inflate areas of the dressing. As the inflatable ring, column or other shape is inflated, the dressing could be removed from the surface of the wound. This mechanism would allow for the dressing to be pulled out of a cavity or distracted in a controlled manner without having to remove or replace the dressing. A one-way value or a screw release valve could allow inflow to inflate the bladder in order to distract or mobilize the dressing against the wound surface or cavity.

Cranks or other mechanism can be designed in order to mobilize the dressing on the wound surface to prevent wound ingrowth. These can facilitate lateral movement or movement in a parallel plane to the wound surface.

Suction can be reversed episodically through the radial tunnels in order to preserve the seal and prevent flow at the edges or throughout the dressing/wound. In this configuration, episodic time periods can be predetermined or scheduled or programed in order to prevent pooling. This can be useful especially in wounds that are vertical. Pooling may occur especially after irrigation at the lower areas of the wound. Suction instead of being directed through the central suction chamber, can be either permanently or temporarily directed through the radial tubing. This reversal of suction pathways can be used to remove any pooling at the most inferior portion of the wound.

### I. Examples of Techniques

1. Reverse Pump Lavage

Applying and removing suction in a specific fashion agitates tissue during wound irrigation and improves wound cleansing and foreign matter/debris removal. Suction is applied to the wound interface component 120 over a time frame to increase vacuum pressure from zero vacuum pressure to a threshold vacuum pressure. The vacuum pressure rate of change varies from −10 mmHg/m to −100 mmHg/m. In some embodiments, short bursts of positive pressure (e.g., pressure above zero mmHg) are applied during the time frame reversing the direction of air flow and thereby varying pressure and increasing wound agitation. Wound interface component 120 positive pressure application is applied with a supplementary pump capable of producing positive pressures to attached vacuum circuits. This supplementary pump also performs pumping functions in the event of EVR 102 pump malfunction.

Alternatively, the EVR 102 could be placed on a reversible flow pathway. A rotating or switching valve manages flow direction creating a bidirectional suction/pumping pathway. Reversing the pumps creates a positive pressure for reverse pulse lavage. Alternatively, sustained positive pressure can be used for use in the tensioner. The positive pressure can be created while sealing the vacuum pressure over the wound in order to utilize a single pump for both devices (e.g., wound interface component 120 and tensioner 116). The EVR 102 controls are used to manage the flow rates, strength of suction, cycling and the direction. Controls or control schemes can be created for irrigation, reverse pulse lavage, tensioning cycling, continuous or intermittent suction for NPWT.

The EVR 102 also regulates supplied positive pressure, or gravity driven flow, for gas or fluid irrigation of any type to the wound interface component 120 and wound. The EVR 102 controls the external positive pressure pump or gravity flow set up. In some embodiments, the EVR 102 includes a positive pressure pump for driving the irrigation/delivery of fluids/gases to the wound interface component 120 and wound.

The EVR 102 utilizes positive pressure to manage a unidirectional bladder, either independent from or as part of the tensioning device (e.g., tensioner 116). The vacuum pressure gradient for the wound interface component 120 is supplied by one of an external pump or the EVR 102 positive pressure pump to create positive pressure pulses for tensioning. The EVR 102 positive pressure pump also creates and maintains a pressure gradient followed by intermittent bursts of positive pressure for other applications.

A rotating mechanism could be designed to allow the lower level of the wound interface component to rotate under the upper layer. In doing this, wound cleaning and debridement could be enhanced. A central axis of rotation could allow for rotation of the disk with the perforated holes and radial tubing to occur in the plane of the wound.

2. Gas Therapy

A hyper-atmospheric (e.g., above atmospheric level, hyper-concentrated, super saturated) concentration of oxygen or other gases can be run over the wound through the wound interface component 120. Nitrous oxide, carbon monoxide as well as other gases could also be used based on their therapeutic mechanisms and the needs of the wound. This process is performed via multiple options. The EVR 102 can be in fluid connection with a gas concentration mechanism (e.g., oxygen concentrator) where gases (e.g., oxygen, hydrogen, nitrogen) can be concentrated to a threshold level in order to tailor the management of the wound. The EVR 102 can create this concentration via the positive pressure pump and delivered to the wound interface component 120. The EVR 102 performs this therapy while controlling the gas concentration and flow rate. The gas can be nebulized or moisturized to prevent the wound from drying out. A static filter (e.g., HEPA filter) can perform these functions via pore size, ionic charge, or other means to prevent wound contamination during gas therapy. The static filter can be included in the vacuum circuit between the gas source and the wound interface component 120 where the gas is delivered free- or substantially free of contamination. The controlled flow of the gas therapy also prevents dead space creation and directs the therapy gas to flow through the wound interface component 120.

A liquid canister (e.g., moisturizer, water, antibiotic fluid, or other liquid therapeutics) mixes the liquid and gas, moisturizing the gas and tailoring the gas therapy to patient or wound needs. The canisters can be disposable or reusable/refillable. In some embodiments of the EVR 102, the EVR 102 controls gas therapy parameters to a set program. For example, the EVR 102 controls the therapeutic liquid release via a liquid regulator, or warming or cooling the therapeutic liquid. The EVR 102 regulates wound temperature via flowing temperature-regulated gases or liquids over the wound thereby increasing (or restricting) blood flow, having the effect of regulating some biological processes such as inflammation, swelling, or apoptosis.

In some embodiments, a large capacity source (e.g., a wall supply, or disposable or refillable canisters such as a pressurized gas tank) supplies the therapy gas. The EVR 102 controls therapy gas flow rate to the wound through pressure regulation. In general, the canisters can include communications components enabling remote access, monitoring, and/or management. The canisters could contain memory capacity to record data. These canisters could be able to refill its storages via air compressors built into the units. These canisters could communicate in a bidirectional manner as well and be interactive on the system or network of devices.

These canisters can contain one or more gases or therapeutics and the EVR 102 flow the canister gas to the wound in a specific time sequence or mixture to tailor the gas therapy to the specific wound or patient. These canisters could contain biological substances.

Alternatively, the canister attaches directly to the wound interface component 120 inflow tubing. The canister contains the pressurized therapy gas or the canister can be externally pressurized to deliver the therapy gas or fluid through the irrigation tubing circuit of an enhanced vacuum pressure wound therapy wound interface component 120 (e.g., wound interface component 120) controllable by twist valve. A gauge displays the level of gas remaining. Additionally, a small Tillable water reservoir can be included to moisturize the gas.

In some embodiments, a gas compressor external to the EVR 102 provides the gas compression or concentration function, such as a COPD (chronic obstructive pulmonary disease) portable oxygen system. In some embodiments, the external gas compressor is wearable, worn at the belt or strapped to the leg/arm or other area. Separate tubing is attached to the inflow tubing of the wound interface component 120.

In some embodiments, the EVR 102 includes a liquid flow meter monitored by the EVR 102 which produces alarms in high flow rate cases such as bleeding. An alarm notification triggers if a flow rate increase is detected without active fluid irrigation. An increased flow time duration during a period of irrigation can be programmed for the EVR 102 thereby preventing alarm triggering during fluid irrigation. This can be 1-time button that is engaged every time irrigation occurs or as a continuous background algorithm.

The EVR 102 can include modes for irrigation or suction. An irrigation mode disarms the flow rate alarm that triggers during a potential active bleed or vacuum assisted exsanguination event. Irrigation parameters such as output, duration, or type, can be monitored and recorded by the EVR 102 to ensure therapeutic activities were performed and in some cases performed as a monitoring means for billing and quality control measures. This tracking feature allows providers additional information when assessing patient response to treatment. If, for instance, the patient does not respond to the prescribed treatment, the provider can confirm the patient has been compliant with the prescribed therapy.

Two pumps can be arranged in circuit with the EVR 102 operating in opposite flow directions, such as a positive pressure pump and a vacuum pressure pump. Alternatively, the EVR 102 pump can be a bi-directional pump, e.g., switchable to operate as a positive or vacuum pump. As an example, a bi-directional pump applies suction followed by positive pressure by switching the direction the pathway is directed.

The EVR 102 pump and canister connection can be magnetic which enables easy fit and connection/disconnection. The connection can further include electrical connections allowing the EVR 102 to receive canister identification information via a microchip or RF signal. The information received from the canister can be utilized to deactivate the pump unless combined with an authorized canister to prevent use without authorized or genuine canisters.

The connection between canister tubing and wound interface component 120 canister can be magnetic and/or electrical, as described above. In some embodiments, a proprietary connection prevents the wound interface component 120 connection to a non-authorized canister and/or EVR 102. The wound interface component 120 can have electrical wiring that provides suction gradient information at least one site thereby detecting whether a leak is occurring. In the absence of an authorized canister connected to the wound interface component 120, the EVR 102 can create a mechanical block or malfunction in the suction tubing circuit thereby preventing use of non-authorized suction, canisters, or pumps. Alternatively, wound interface component 120 and suction canisters can utilize microchips containing identification information, thereby allowing recognition of authorized devices.

Control of the materials input into the system can be controlled by proprietary connectors, or microchips or RFID that signal to the EVR to allow the intervention. It would also serve to identify the intervention and ensure it is safe to do so at that time. Combinations of some gases and chemicals may result in unsafe combinations. Disposable small canisters used in paintball guns, Nail guns . . . could be designed to fit directly onto the inflow tubing. Regulated flow plus or minus moisturizing of the gas would be predetermined in order to provide a specified amount of gas over a specific time interval at a specified flow rate.

3. UV-Light Bacterial Count Measurements

The EVR 100 includes spectroscopic components to detect fluorescently-labeled antibody probes or similar biologic labeling methods that can bind to selected markers in the wounds. For example, bacterial cell wall proteins or specific biomolecules that indicate healing or unhealthy wound healing progress. The unified construction of the wound interface component 120 and sealing layer are composed of substantially transparent materials thereby allowing light emitted from bound probes at the wound surface to be detected by external wound interface component components. The wound interface component 120 includes photo-sensing devices to measure emitted light and algorithms to quantify detected information such as bacterial bioburden.

The wound interface component 120 irrigation system serves as a probe delivery mechanism. The wound interface component 120 can regulate vacuum applied to the wound. For example, after a period of time with no applied vacuum pressure, the wound interface component 120 reapplies vacuum pressure. Alternatively, the wound interface component 120 regulates the flow of irrigant containing the probes during lavage flow across the entire wound surface depending on binding kinetics of the probe.

The photo-sensing device adjacent the wound surface can be portable and user operated (e.g., hand-held) or stationary (e.g., mounted to the wound interface component 120). In some embodiments, the photo-sensing device can be operated for spot checks (e.g., single time points) or run continuously, depending on the provider-determined intervals, considering rate of change in the probe targeted ligand or substrate. Emitted light can be measured after instillation of probing agent or it can be measured over time after an instillation to determine the rate of decay of the probe signal. The independent variables effecting the amount of emitted light signal and the methods for measuring and interpreting this light can be controlled to achieve specific uses. In some embodiments, the unified construction of the wound interface component 120, allows for the photo-sensing monitor to be incorporated into the wound interface component 120 with either hard-wired or blue-tooth communication to the EVR 102.

The EVR 102 stores in memory algorithms to quantify bacterial bioburden based upon received light signals and alarm notifications based on rate of rise or absolute total amount threshold values of detected bioburden. The algorithms use the threshold values to enable bacterial management devices, such as enabling one or more UV light source described above, or initiating fluid or gas (such as oxygen or chloride) irrigation. Wound interface component irrigation tubing includes ports for connection of ampules containing therapeutic materials, such as antibiotics in preset doses, for dispensing to the wound surface. Additionally, once detected bioburden values decrease beneath low value thresholds, biologics, such as stem cells, can be released via the same mechanism to increase healing.

4. Pressure Based Ulcer Prevention and Management

Pressure sores can occur due to thin tissue with limited soft tissue over boney prominences. The barrier 802 can be expanded in depth to allow for not only eliminating in-growth as well as padding to prevent pressure sores. In some embodiments, the wound interface component barrier 802 is slightly compressible (e.g., soft) and porous, functioning as a fluid sponge or shock absorber. More than one barrier 802 can be stacked or layered to provide improved padding. Alternatively, the barrier 802 is constructed to include a thicker barrier 802 layer (e.g., >5 mm) thereby supplying additional cushioning to the wound. Optionally, air bladders are built into the barrier 802 or unified wound interface component 120 that can be episodically inflated to provide cushioning as well as improved circulation.

Specific designs for unified wound interface component 120 as well as the barrier 802 built into the wound interface component 120 include wound interface component 120s including hydrogel bumpers in concentric rings providing additional cushioning. These rings can be on the dorsal (away from the wound) or volar side (on the wound surface) of the wound interface component 120.

These paddings can be designed for specific areas of the body. For example, socks for posterior heel pads including barrier 802 and padding protection. The wound interface component 120 and padding can include an adhesive surface for adhesion to skin or wound surfaces. As a second example, pants including barrier 802 and padding protection for sacral wounds. In some embodiments, the barriers 802 prevent wound in-growth. In some embodiments, the wound interface components 120 incorporate pneumatic bladders for padding or improved circulation.

5. Pain Management

The system 100 can include a pain-relief pump delivering local anesthesia in a specific area, such as a nerve, for extended pain relief. Additionally, anesthesia can be administered through the unified wound interface component 120 in order to reduce pain sensations. The anesthesia is delivered through suction irrigation tubes to deliver pain relief to the subcutaneous or intramuscular or the periwound tissue for pain management. The delivery of ampules of medicine can be controlled via the EVR 100 as described herein.

6. Peritoneal Dialysis

The unified wound interface component 120 could be used for temporary dialysis for patients with renal insufficiency or failure. The wound interface component 120 could be placed inside the intra-abdominal cavity and inflow used to dispense dialysis fluid into the abdominal cavity. The outflow could be used to remove fluid once diffusion occurs. This can be done over a period of time or continual as the needs of the patient require.

7. Controlled Tissue In-Growth

In some embodiments, the wound interface component 120 includes a layer of a powder coated material (e.g., TPE/TPU/Polymer/Silicone) including small pore sizes (e.g., about 40 nm) similar to a sponge. This layer creates a contact surface of a depth between 1 mm and 5 mm for the wound interface component 120 at the wound surface allowing a removable ingrowth depth (e.g., debridement) similar to a wet or dry fabric wound interface component 120 wherein changing the wound interface component 120 removes the top wound layer including any dead or foreign matter. Alternatively, the pore size is arranged in a pattern that does not allow for free particles to be left behind. In-growth can additionally be promoted via a separate material such as suture, metallic abrasive pad, or sponge. The wound interface component 120 allows in-growth with a planned wound interface component 120 change at 2-3 days to a long-term wound interface component 120 without in-growth capabilities.

A screen is built into the barrier allowing limited in-growth through the perforations on the barrier allowing for the wound interface component 120 to be cut and the perforated sheet polymer material preventing particle deposition. Screen thickness can be between 1 cm to 4 cm to limit the depth of in-growth.

8. Military Applications

Some embodiments of the systems disclosed herein allow for portable frames that collapse into small storage sizes but open and lock into larger, rigid frames. These frames can vary in size based on the needs. Additionally, collection bags can then be used and even reused in order to separate fluid from gas. Portable frames for holding EVR system components can be designed to limit space for military use. The portable frames can be constructed from collapsible components, for example tent poles or collapsible cups. The wound interface component canister can be reused or disposed of after use thereby limiting packaging storage space in personal carrying vessels, such as a backpack.

The portable frame includes one or more fasteners, e.g., latches, to temporarily secure the structure into a rigid position. Releasing this fastener allows the device/structure to reversibly collapse.

In some embodiments, the frame functions to support a suction canister or irrigation fluid collection. The collection frame includes bags (e.g., plastic or other material) fitted to secure to the rigid frame preventing bag collapse to allow use in a mechanical wound therapy system.

Referring to FIG. 10, in low- or no-power availability situations, components of the system 100 can be fluidly connected and used in an alternative configuration. For example FIG. 10 depicts an unpowered configuration including fluid collection and filtration system 200, unified wound interface component 120, and non-powered pump 400.

9. Temperature Regulation

Patient temperature can be regulated at a local (e.g., wound) or systemic (e.g., core) level using the system 100. In some embodiments, the unified wound interface component 120, or the adhesive layer thereof, includes a closed tubing system. The closed tubing system is constructed into the layered wound interface component 120 for circulating temperature-regulated gases or fluids through the wound interface component 120 without touching the wound. For example, the closed tubing system can be arranged in a radial coil, or zig-zag pattern (e.g., back and forth) over the wound surface.

Compressed gas when allowed to expand provides cooling. Small canisters can be used to allow gas expansion in order to cool the wound surface in times when reduced temperature can assist in reducing swelling or improving healing.

The unified wound interface component 120 including a closed tubing system can regulate patient or wound temperatures for short durations (e.g., <1 hr.) or prolonged durations (e.g., >1 hr.). Depending on the temperature of the temperature-regulated gases or fluids, the patient or wound can be heated or cooled. In some embodiments, the patient or wound temperature can be alternated between heated and cooled. This technique manages patient temperature in a non-medical setting (e.g., military, camping, remote) to treat or prevent hypothermia or heat exhaustion in the absence of a wound.

In some embodiments, the closed tubing system is an independent layer disposed over the top of the barrier 802. In further embodiments, the closed tubing system is integrated with the hydrocolloid adhesion layer.

As an example of unpowered cooling using the closed tubing system, compressed CO2 (e.g., from a disposable canister or refillable tank) can be released through the system. As the gas expands, the gas cools thereby removing heat from the surrounding environment. Alternatively, an exothermic reaction could be used to create of an unpowered heating system.

10. Windowed Wound Interface Component

A common clinical practice to obtain a better seal in NPWT is to place a highly adhesive layer (e.g., hydrocolloid sheet) over top of a wound. The sheet covers the wound and periwound completely and forms a positive seal on the skin. An opening (e.g., a window) is cut into the sheet that corresponds to the size and location of the wound. This is termed “windowing” a wound interface component. The standard NPWT wound interface component 120 is placed over the wound interface component window and the wound interface component 120 sealing drape is attached to the periphery of the initially placed adhesive layer. This technique provides protecting for delicate skin in the setting of the hydrocolloid adhesive layer.

The wound interface component 120 sealing layer may be standard drape material, instead of the hydrocolloid, which removes from the top of the windowed hydrocolloid layer without disrupting wound. The windowed hydrocolloid layer is used through multiple wound interface components 120 changes and eventually removed using adhesive remover or as the top layer of skin sloughs off.

Alternatively, the periwound could also be treated with a paintable adhesive, or “new skin”, is placed around the wound to improve the seal or protect the skin. The paintable adhesive prevents adhesion to the skin by the hydrocolloid. In some embodiments, the paintable adhesive is dissolvable using a solvent to remove the adhesive layer.

11. Veterinary Uses

As described herein, the EVR system can be used with human patients. However, the system can be adapted for non-human subjects and maintain similar function. For example, wound management of livestock, small animals, large animals, pets, exotic animals, reptiles, or marine animals can be performed.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable sub-combination. Moreover, although features may be described herein as acting in certain combinations and/or initially claimed as such, one or more features from a claimed combination can in some cases be excised from the combination, and the claimed combination may be directed to a sub-combination or variation of a sub-combination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

A number of embodiments of the inventions have been described. Nevertheless, it will be understood that various modifications can be made without departing from the spirit and scope of the invention. For example, in some embodiments various components such as radiopaque material, filaments, flow passages, etc. need not be included. Moreover, the shape of various features of the barrier can be modified as appropriate. Furthermore, while some embodiments are disclosed in combination with NPWT, many of the features disclosed herein can be used either independently of NPWT (i.e. in a wound care system configured for drug delivery without NPWT) or in conjunction with NPWT. Accordingly, other embodiments are within the scope of the following claims.

12. Seal Improvement

Seal improvement can be obtained through multiple options. A spray or gel or paste can be used to improve seals. Benzoin, mastisol or other skin preps can be used. Hydrocolloid or hydrogels or silicone-based adhesives can be used. These preps can be used to assist in prolonging or improving the seal.

What is claimed is:

1. A mechanical wound therapy system, comprising:
a wound interface component configured to be positioned adjacent to a wound;
a vacuum source configured to generate a suction force that produces a negative pressure differential nearby the wound;
an inflow component fluidly coupled to the wound interface component and the vacuum source;
a vacuum regulator device fluidly coupled to the vacuum source, wherein:
the suction force generated by the vacuum source is regulated, and
a set of parameters associated with the regulated suction force is monitored.

2. The system of claim 1, further comprising a tensioning device configured to be placed adjacent to the wound.

3. The system of claim 1, wherein the vacuum regulator device comprises:
a microprocessor that regulates the suction force generated by the vacuum source and monitors the set of parameters associated with the regulated suction force; and
a communication module configured to transmit, for output, data representing the set of parameters monitored by the processor.

4. The system of claim 3, wherein:
the communication module comprises a near-field communication module; and
the near-field communication module is configured to:
establish a short-range connection with a computing device that is within a proximity to the apparatus, and
transmit, over the short-range connection, the data representing the parameters to the computing device.

5. The system of claim 3, wherein the communication module comprises a Wi-Fi module.

6. The system of claim 3, wherein the communication module or encrypts or otherwise secures the information being transmitted.

7. The system of claim 5, wherein the Wi-Fi module is configured to:
connect to a local area network; and
transmit, over the local area network, the data representing the parameters to a computing device connected to the local area network.

8. The system of claim 5, wherein the Wi-Fi module is configured to:
connect to a wide area network; and
transmit, over the wide area network, the data representing the parameters to a server that is remote from the apparatus.

9. The system of claim 1, wherein regulation of the suction force applied by the vacuum source is programmable by a user.

10. The system of claim 1, wherein the wound interface component, the vacuum source, and the vacuum regulator each comprise circuitry configured to be in data communication with a remote monitoring system.

11. The system of claim 10, wherein the circuitry of each of the wound interface component, the vacuum source, and the vacuum regulator is configured to receive error data via a wireless signal to the remote monitoring system.

12. The system of claim 1, wherein the wound interface component, the vacuum source, and the vacuum regulator each comprise at least one sensor.

13. The system of claim 10, and further comprising:
an exudate canister fluidly coupled between the wound interface component and the vacuum source, wherein the exudate canister comprises circuitry configured to be in data communication with the remote monitoring system.

14. The system of claim 1, and further comprising a remote monitoring system.

15. The system of claim 1, wherein the vacuum source comprises a portable vacuum.

16. The system of claim 1, wherein the vacuum source comprises a wall vacuum.

17. A mechanical wound therapy system comprising:
a dressing comprising a top layer and a bottom layer, wherein:
the dressing is configured to be positioned adjacent to a wound,
the bottom layer is positioned to face the wound and includes a set of perforations;
a vacuum source configured to generate a suction force that produces a negative pressure differential nearby the wound; and
a regulator device fluidly coupled to the mechanical wound therapy system, wherein the regulator device is configured to:
regulate the suction force generated by the vacuum source, and
monitor a set of parameters associated with the regulated suction force.

18. The system of claim 17, wherein the regulator device comprises:
a microprocessor that regulates the suction force generated by the vacuum source and monitors the set of parameters associated with the regulated suction force; and
a communication module configured to transmit, for output, data representing the set of parameters monitored by the processor.

19. The system of claim 18, wherein:
the communication module comprises a near-field communication module; and
the near-field communication module is configured to:
establish a short-range connection with a computing device that is within a proximity to the apparatus, and
transmit, over the short-range connection, the data representing the parameters to the computing device.

20. The system of claim 18, wherein the communication module comprises a Wi-Fi module.

21. The system of claim 18, wherein the communication module or encrypts or otherwise secures the information being transmitted.

22. The system of claim 20, wherein the Wi-Fi module is configured to:
connect to a local area network; and
transmit, over the local area network, the data representing the parameters to a computing device connected to the local area network.

23. The system of claim 20, wherein the Wi-Fi module is configured to:
connect to a wide area network; and
transmit, over the wide area network, the data representing the parameters to a server that is remote from the apparatus.

24. The system of claim 17, wherein regulation of the suction force applied by the vacuum source is programmable by a user.

25. The system of claim 17, wherein the dressing, the vacuum source, and the regulator device each comprise circuitry configured to be in data communication with a remote monitoring system.

26. The system of claim 25, wherein the circuitry of each of the dressing, the vacuum source, and the regulator device is configured to receive error data via a wireless signal to the remote monitoring system.

27. The system of claim 17, wherein the dressing, the vacuum source, and the regulator device each comprise at least one sensor.

28. The system of claim 17, wherein the bottom layer of the dressing is composed of plastic and includes a set of perforations.

29. The system of claim 17, wherein the bottom layer of the dressing is composed of a thermoplastic elastomer and includes a set of perforations.

30. A vacuum regulator apparatus for wound therapy, the apparatus comprising:
an interface configured to be coupled to a vacuum source such that the vacuum applies a suction force to a wound when coupled to the interface;
a processor configured to:
regulate the suction force applied by the vacuum; and
monitor a set of parameters associated with the suction force applied by the vacuum; and
a communication module configured to transmit, for output, data representing the set of parameters monitored by the processor.

31. The apparatus of claim 30, wherein the vacuum regulator is configured to be programmed by a user for regulation of the suction force applied by the vacuum source.

32. The apparatus of claim 30, wherein the set of parameters associated with the suction force applied by the vacuum source comprises at least one user-specified parameter.

33. The apparatus of claim 30, further comprising a rechargeable battery configured to power the processor and the communication module.

34. The apparatus of claim 30, wherein:
the communication module comprises a near-field communication module; and
the near-field communication module is configured to:
establish a short-range connection with a computing device that is within a proximity to the apparatus, and
transmit, over the short-range connection, the data representing the parameters to the computing device.

35. The apparatus of claim 30, wherein the communication module comprises a Wi-Fi module.

36. The apparatus of claim 35, wherein the Wi-Fi module is configured to:
connect to a local area network; and
transmit, over the local area network, the data representing the parameters to a computing device connected to the local area network.

37. The apparatus of claim 35, wherein the Wi-Fi module is configured to:
connect to a wide area network; and
transmit, over the wide area network, the data representing the parameters to a server that is remote from the apparatus.

38. The apparatus of claim 30, wherein the communication module is configured to exchange bi-directional communications with one or more components of a negative pressure wound therapy (NPWT) system.

39. The apparatus of claim 38, wherein the one or more components comprises a wound interface component, an irrigation network, or an exudate cannister.

40. The apparatus of claim 30, further comprising a storage device configured to store data representing the set of parameters.

41. The apparatus of claim 30, wherein:
the processor is configured to monitor device usage during a rental period for the vacuum regulator apparatus; and
the communication module is configured to transmit, for output to a billing system, data representing monitored usage of the vacuum regulator apparatus during the rental period.

42. The apparatus of claim 30, wherein:
the processor is configured to:
detect that the vacuum regulator apparatus has been turned on and being used for negative wound therapy, and
in response to detecting that the vacuum regulator apparatus has been turned on and being used for negative wound therapy, collect data indicating a patient identifier associated with the negative round therapy; and
the communication module is configured to transmit data representing the patient identifier for output to a billing system.

43. The apparatus of claim 30, further comprising:
a microphone configured to collect utterances provided by a user; and
the processor is configured to:
process the utterances collected by the microphone to identify a voice query corresponding to the processed utterance, and
generate an instruction to perform an operation based on the identified voice query.

44. The apparatus of claim 30, further comprising a set of interface controls for adjusting settings for providing negative wound therapy to the wound.

45. The apparatus of claim 44, wherein the set of interface controls comprises for providing negative wound therapy to the wound.
